(19) United States
(12) Patent Application Publication
(10) Pub. No.: US 2002/0119945 A1
(43) Pub. Date: Aug. 29, 2002

(54) Methods for inhibiting proliferation of astrocytes and astrocytic tumor cells and uses thereof

(76) Inventor: David E. Weinstein, Dobbs Ferry, NY (US)

Correspondence Address: Craig J. Arnold, Esq., Amster, Rothstein & Ebenstein, 90 Park Avenue, New York, NY, 10016, US

(21) Appl. No.: 10/035,914

(22) Filed: Nov. 7, 2001

Related U.S. Application Data
(60) Provisional application No. 60/246,868, filed on Nov. 8, 2000.

(51) Int. Cl. A61K 48/00; C12N 15/85; C12N 15/87; C12N 15/88

(52) U.S. Cl. 514/44; 435/455; 435/458; 435/459

(57) ABSTRACT

The present invention provides methods for inhibiting proliferation of astrocytes and astrocytic tumor cells. The present invention further provides methods for treating a condition associated with a defect in astrocyte proliferation in a subject, and methods for treating a condition associated with astrocytic tumor cell proliferation in a subject. Additionally, the present invention is directed to pharmaceutical compositions comprising CD81 protein or nucleic acid and a pharmaceutically-acceptable carrier. Finally, the present invention provides a method for determining whether a subject has an astrocytoma, and a method for assessing the efficacy of astrocytoma therapy in a subject.

### RELATED APPLICATION

This application claims the benefit of U.S. Provisional Application No. 60/246,868, filed Nov. 8, 2000.

### BACKGROUND OF THE INVENTION

This year, and each year in the foreseeable future, 17,000 people in the United States will be diagnosed with brain tumors. The majority of these tumors will be of astrocyte lineage, and most people diagnosed with these malignancies will die of their diseases. Brain tumors, or intracranial neoplasms, are found in about 2% of all routine autopsies. They are most common in early or middle adult life, but may occur at any age. Their frequency also appears to be increasing in the elderly (31).

Brain tumors invade and destroy normal tissue, producing such effects as impaired sensorimotor and cognitive function, increased intracranial pressure, cerebral edema, and compression of brain tissue, cranial nerves, and cerebral vessels (31). Metastases may involve the skull or any intracranial structure. The size, location, rate of growth, and histologic grade of malignancy determine the seriousness of brain tumors. Nonmalignant tumors grow slowly, with few mitoses, no necrosis, and no vascular proliferation. Malignant tumors grow more rapidly, and invade other tissues. However, they rarely spread beyond the central nervous system (CNS), because they cause death by local growth. Drowsiness, lethargy, obtuseness, personality changes, disordered conduct, and impaired mental faculties are the initial symptoms in 25% of patients with malignant brain tumors (31).

While brain tumors, or intracranial neoplasms, are common, they are frequently misdiagnosed (31). Brain tumors may be classified by site (e.g., brain stem, cerebellum, cerebrum, cranial nerves, ependyma, meninges, neuroglia, pineal region, pituitary gland, and skull) or by histologic type (e.g., meningioma, primary CNS lymphoma, or astrocytoma) (31). Common primary childhood tumors are cerebellar astrocytomas and medulloblastomas, ependymomas, gliomas of the brain stem, and congenital tumors. In adults, primary tumors include meningiomas, schwannomas, and gliomas of the cerebral hemispheres (particularly the malignant glioblastoma multiforme and anaplastic astrocytoma, and the more benign astrocytoma and oligodendroglioma). Overall incidence of intracranial neoplasms is essentially equal in males and females, but cerebellar medulloblastoma and glioblastoma multiforme are more common in males (31).

Gliomas are tumors composed of tissue representing neuroglia in any one of its stages of development (31). They account for 45% of intracranial tumors. Gliomas can encompass all of the primary intrinsic neoplasms of the brain and spinal cord, including astrocytomas, ependymomas, and neurocytomas. Astrocytomas are tumors composed of transformed astrocytes, or astrocytic tumor cells. Such tumors have been classified in order of increasing malignancy: Grade I consists of fibrillary or protoplasmic astrocytes; Grade II is an astroblastoma, consisting of cells with abundant cytoplasm and two or three nuclei; and Grades III and IV are forms of glioblastoma multiforme, a rapidly growing tumor that is usually confined to the cerebral hemispheres and composed of a mixture of astrocytes, spongioblasts, astroblasts, and other astrocytic tumor cells. Astrocytoma, a primary CNS tumor, is frequently found in the brain stem, cerebellum, and cerebrum. Anaplastic astrocytoma and glioblastoma multiforme are commonly located in the cerebrum (31).

Treatment of brain tumors is often multimodal, and depends on pathology and location of the tumors (31). For malignant gliomas, multimodal therapy, including chemotherapy, radiation therapy, and surgery, is used to try to reduce tumor mass. Regardless of approach, however, prognosis for patients suffering from these tumors is guarded: the median term of survival after chemotherapy, radiation therapy, and surgery is only about 1 year, and only 25% of these patients survive for 2 years. In view of the foregoing, it is imperative that new ways be developed for diagnosing, detecting, and treating malignant gliomas (31).

Astrocytes also have been implicated in pathologies produced by virtually all neural traumas, including CNS injury and neuronal cell death resulting from neurodegenerative disease. In the case of CNS injury, for example, resulting astrocytosis is thought to be a major contributor to the formation of a glial scar, which is believed to present a major barrier to productive neural regeneration (6). Therefore, a primary goal in the design of therapeutics for both CNS trauma and neurodegenerative diseases is the elucidation of mechanisms for limiting glial scar formation.

Head injuries cause more deaths and disability than any other neurologic condition before age 50, and occur in more than 70% of accidents—the leading cause of death in men and boys less than 35 years of age. Mortality from severe injury approaches 50%, and is only modestly reduced by treatment. Damage may result from skull penetration or from rapid brain acceleration or deceleration, resulting in injury to surrounding tissue. Currently, there is no treatment for astrocytosis resulting from head trauma.

Alzheimer's disease is a neurodegenerative disease characterized by a progressive, inexorable loss of cognitive function (31). The pathogenesis of Alzheimer's disease is associated with an excessive number of neuritic, or senile, plaques (composed of neurites, astrocytes, and glial cells around an amyloid core) in the cerebral cortex, and neurofibrillary tangles (composed of paired helical filaments). Approximately 4 million Americans suffer from Alzheimer's disease, at an annual cost of about $90 billion. The disease is about twice as common in women as in men, and accounts for more than 65% of the dementias in the elderly. While senile plaques and neurofibrillary tangles occur with normal aging, they are much more prevalent in persons with Alzheimer's disease. To date, a cure for Alzheimer's disease is not available, and cognitive decline is inevitable.

At present, there are no specific treatments for astrocytosis. In addition, while there are standard chemotherapeutic, radiotherapeutic, and surgical treatments for astrocytoma, these therapies are fraught with severe limitations, and are often palliative rather than curative. Accordingly, there is a great need to develop methods of treating astrocytomas, astrocytosis, and other conditions associated with a proliferation of astrocytes or astrocytic tumor cells. An understanding of the basic biology of neuron-glial interaction may provide insight into the elucidation of such treatment options.

### SUMMARY OF THE INVENTION

The present invention is based upon the discovery that CD81 modulates proliferation of astrocytes in neural tissue, and is not expressed in astrocytic tumor cells. On the basis of this finding, the present invention provides a method for inhibiting proliferation of astrocytes, by contacting astrocytes with an amount of CD81 effective to inhibit proliferation of astrocytes.

The present invention further provides a method for treating a condition associated with a defect in astrocyte proliferation in a subject in need of treatment, by contacting astrocytes in the subject with an amount of CD81 effective to inhibit proliferation of astrocytes, thereby treating the condition. Also disclosed is a method for inhibiting proliferation of astrocytes, by contacting astrocytes with a modulator of CD81 expression in an amount effective to induce or enhance expression of CD81, thereby inhibiting proliferation of astrocytes.

The present invention further provides a method for inhibiting proliferation of astrocytic tumor cells, by contacting astrocytic tumor cells with an amount of CD81 effective to inhibit proliferation of astrocytic tumor cells. Additionally, the present invention discloses a method for treating a condition associated with proliferation of astrocytic tumor cells in a subject in need of treatment, by contacting astrocytic tumor cells in the subject with an amount of CD81 effective to inhibit proliferation of astrocytic tumor cells, thereby treating the condition. The present invention is further directed to a method for inhibiting proliferation of astrocytic tumor cells, by contacting astrocytic tumor cells with a modulator of CD81 expression in an amount effective to induce or enhance expression of CD81, thereby inhibiting proliferation of astrocytic tumor cells.

The present invention also provides a method for treating a condition associated with a defect in astrocyte proliferation in a subject in need of treatment, by administering to the subject an amount of CD81 effective to treat the condition associated with a defect in astrocyte proliferation. Also disclosed is a method for treating a condition associated with proliferation of astrocytic tumor cells in a subject in need of treatment, by administering to the subject an amount of CD81 effective to treat the condition associated with proliferation of astrocytic tumor cells.

The present invention further provides a method for treating a condition associated with a defect in astrocyte proliferation in a subject in need of treatment, by administering to the subject a modulator of CD81 expression in an amount effective to induce or enhance expression of CD81, thereby treating the condition associated with a defect in astrocyte proliferation in the subject. Also provided is a method for treating a condition associated with proliferation of astrocytic tumor cells in a subject in need of treatment, by administering to the subject a modulator of CD81 expression in an amount effective to induce or enhance expression of CD81, thereby treating the condition associated with proliferation of astrocytic tumor cells in the subject.

Additionally, the present invention is directed to pharmaceutical compositions, comprising CD81 and a pharmaceutically-acceptable carrier, or comprising nucleic acid encoding CD81 and a pharmaceutically-acceptable carrier.

The present invention further provides a method for determining whether a subject has an astrocytoma, by assaying for CD81 expression a diagnostic sample of cells of astrocytic lineage of the subject, wherein no detection of expression of CD81 in cells of astrocytic lineage of the subject is diagnostic of an astrocytoma.

Finally, the present invention is directed to a method for assessing the efficacy of astrocytoma therapy in a subject who has undergone or is undergoing treatment for an astrocytoma, by assaying for CD81 expression a diagnostic sample of cells of astrocytic tumor cells of the subject, wherein no detection of expression of CD81 in astrocytic tumor cells of the subject is indicative of unsuccessful astrocytoma therapy.

Additional objects of the present invention will be apparent in view of the description which follows.

### BRIEF DESCRIPTION OF THE FIGURES

FIGS. 1A-1C demonstrate that CD81 is expressed on the surface of the astrocyte. After the expression of CD81 message was identified by differential screen, protein expression was determined by Western blot analysis. (A) While astrocytes express ample CD81, the C6 glioma cell line is CD81-negative. To localize the protein expression in the astrocyte, astrocytes were cultured either alone (B) or in the presence of neurons (C) for 48 h, then fixed and stained for the expression of CD81 on the cell surface, using the monoclonal antibody 2F7. The arrows in (B) point out the expression of CD81 along the astrocyte processes—a domain of the cell critical for neuronal interaction (10).

FIGS. 2A-2H illustrate that CD81 is a critical mediator of neuron-astrocyte interactions. Eat1 effectively interfered with normal neuron-mediated astrocyte proliferation. (A) In the presence of increasing concentrations of Eat1 monoclonal antibody (mAB), there was a loss of neuron-mediated astrocyte proliferative arrest (closed bars); the antibody had no effect on astrocyte proliferation in the absence of neurons (open bars). (B) In contrast, 2F7 augmented neuronally-induced astrocyte proliferative arrest, such that, in the presence of this mAb and neurons, astrocyte proliferation was further reduced over the level seen with neurons alone. (C) Immunofluorescence studies of astrocytes stained with anti-GFAP antiserum in neuron-astrocyte co-cultures, in the presence of Eat1, showed a dependence on the Eat1 epitope for normal responsiveness to neuronally-induced, astrocyte process formation. In contrast, blocking the Eat2 epitope had no effect on astrocyte responsiveness to neurons, as evidenced by the complex GFAP processes seen in these cultures (D) which had the same appearance as control co-cultures (E). While mAbs Eat1 and 2F7 had profound effects on astrocytic responses to neurons, they had no observable effects on neuronal survival or axonogenesis. Neuron-astrocyte co-cultures also were stained with the mAb TuJ1, which recognizes a neuron-specific βIII tubulin isoform (3). The extent and quality of neurites were comparable in the presence of Eat1 (F) and 2F7 (G), and in control co-cultures (H).

FIGS. 3A-3C demonstrate that GST-CD81 binds to neurons, not astrocytes. Highly-enriched cultures of either neurons or astrocytes from P4 rat cerebellum were established, as described (28). The cells, which were plated at equivalent densities, were cooled on ice, to prevent internalization, then incubated with 10 μg/ml of bacterially-expressed GST-CD81 for 1 h. The cells were fixed, and stained with a goat anti-GST antibody, followed by an Alexia red conjugated rabbit anti-goat secondary. FIG. 3A shows the absence of background staining (no primary antibody control). FIG. 3B shows the binding of the CD81 fusion protein to the surface of the neuron. In contrast, only the few contaminating neurons in the astrocyte-enriched fraction bound the fusion protein (C).

FIG. 4 illustrates that soluble CD81 competes with astrocyte-expressed CD81, and blocks neuron-induced astrocyte quiescence. Increasing concentrations of soluble GST-CD81 were added to co-cultures of neurons and astrocytes. The GST-CD81 competed for neurons with the expressed CD81, thereby blocking neuronal CD81-receptor binding at the astrocyte cell surface. As a result of this competition, astrocytes remained in the cell cycle. 40-50% of neuron-induced inhibition of astrocyte proliferation was achieved with as little as 1 μg/ml of GST-CD81. Maximal inhibition was obtained with 3 μg/ml of soluble protein. The soluble proteins had no observable effects on astrocyte proliferation in the absence of neurons. The specificity of the effect of GST-CD81 was verified by the addition of another, irrelevant GST fusion protein, GST-SCIP, which had no effect on neuron-induced astrocyte quiescence at any concentration tested. Statistical analysis was carried out using the two-tailed students' t-test.

FIG. 5 shows that CD81 is required for neuron-induced astrocyte growth regulation. Mixed cultures of wild-type, CD81+/−, or CD81−/− cerebellar astrocytes and granule cell neurons were established, and evaluated for the role of endogenous CD81 in neuron-induced astrocyte responses. Astrocyte proliferation was determined by double labeling for GFAP and BrdU 48 h after the cultures were established, as described below. Astrocyte proliferation in wild-type co-cultures was determined, and arbitrarily set at 1. CD81 haplo-insufficient astrocytes showed a slight loss of neuron responsiveness (20%). However, astrocytes null at the CD81 locus lost all responsiveness to neurons under these conditions, doubling in number within 48 h after explantation. Assays were done in triplicate for each animal tested, and the overall experiment was repeated three times.

FIG. 6 illustrates that CD81 RNA is absent from a range of astrocytic tumor cell lines. A hallmark of cell transformation is a loss of proliferative arrest in response to naturally-occurring cues. To determine if astrocytic tumor cell lines expressed altered levels of CD81, the inventor extracted RNA from a variety of cell lines, and performed Northern blot analysis. Not unexpectedly, CD81 message was found in wild-type astrocytes. The CD81 levels increased by approximately two-fold when the cells were co-cultured for 48 h with neuronal membranes, suggesting a positive feed-back mechanism affecting CD81 expression. In stark contrast, none of the tumor cell lines tested had any detectable CD81 message, even when the blot was overexposed (not shown). These astrocytic tumors tested were rat: C6 and 9L; human: A172 and U251MG; and mouse: LN308 and LN18. An 18S probe was used as a loading control for RNA.

FIG. 7 depicts the nucleotide sequence of human CD81.

FIG. 8 depicts the amino acid sequence of human CD81.

### DETAILED DESCRIPTION OF THE INVENTION

The present invention provides a method for inhibiting proliferation of astrocytes, by contacting astrocytes with an amount of CD81 effective to inhibit proliferation of astrocytes. Unless otherwise indicated, “CD81” includes a CD81 protein (p27), a CD81 analogue, and a CD81 derivative.

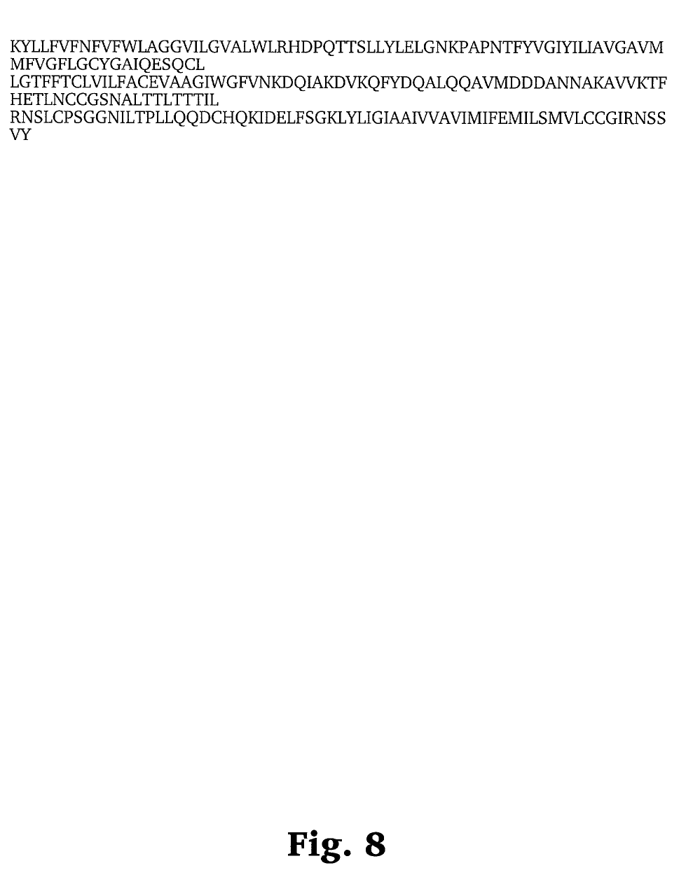

As used herein, CD81 protein has the amino acid sequence set forth in FIG. 8. A “CD81 analogue”, as defined herein, is a functional variant of the CD81 protein, having CD81-protein biological activity, that has 60% or greater (preferably, 70% or greater) amino-acid-sequence homology with the CD81 protein, as well as a fragment of the CD81 protein having CD81-protein biological activity. As further used herein, the term “CD81-protein biological activity” refers to protein activity which modulates and inhibits proliferation of astrocytes and astrocytoma cells, as disclosed herein. Additionally, as used herein, a “CD81 derivative” is a chemical substance derive from CD81, either directly or by modification, truncation, or partial substitution. For example, the CD81 derivative for use in the present invention may be the extracellular domain (ECD) of CD81. In addition, the CD81 derivative of the present invention may be mimetics of CD81, as well as retroinverso versions of these mimetics, in which the D-amino acids are in reverse or inverse orientations.

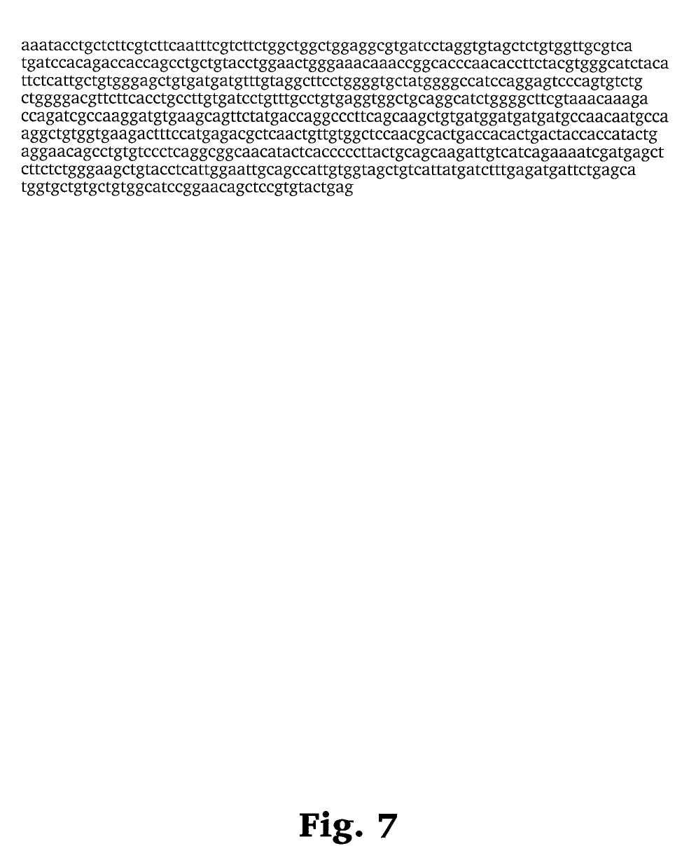

CD81 and its analogues and derivatives may be produced synthetically or recombinantly, or may be isolated from native cells; however, they are preferably produced synthetically, using conventional techniques and cDNA encoding CD81 (FIG. 7). In one embodiment of the present invention, the astrocytes are undifferentiated, i.e., they are not in cell-cycle arrest, and they have not formed complex processes.

The method of the present invention may be used to inhibit proliferation of astrocytes in vitro, or in vivo in a subject. As used herein, the term “inhibit proliferation of astrocytes” means inhibit cell division and growth of astrocytes, and includes limiting the proliferative rate of astrocytes, as disclosed herein. Inhibition of the growth and proliferation of astrocytes may be detected by known procedures, including any of the methods, molecular procedures, and assays disclosed herein.

In accordance with the methods of the present invention, CD81 may be contacted with astrocytes in vitro, or in vivo in a subject, by introducing the CD81 protein into the membranes of astrocytes, or by introducing into the astrocytes a nucleic acid encoding CD81 in a manner permitting expression of CD81 protein. The subject may be any animal, but is preferably a mammal (e.g., humans, domestic animals, and commercial animals). More preferably, the subject is a human. The astrocytes may be contained in neural tissue and other tissue of the nervous system of the subject, either alone or with other types of neural cells, including, for example, neurons and oligodendroglia. Astrocytes may be detected in tissue of the subject by standard detection methods readily determined from the known art, examples of which include, without limitation, immunological techniques (e.g., immunohistochemical staining), fluorescence imaging techniques, and microscopic techniques.

CD81 protein may be introduced into the membranes of astrocytes, either in vitro or in vivo in a subject, by known techniques used for the introduction of proteins into cell membranes (e.g., by means of micro-encapsulated preparations, such as liposomes). The amount of CD81 protein to be used is an amount effective to inhibit proliferation of astrocytes, as defined above, and may be readily determined by the skilled artisan.

For introduction of CD81 protein by way of liposome delivery, liposomal vesicles may be prepared by various methods known in the art, and liposome compositions may be prepared using any one of a variety of conventional techniques for liposome preparation known to those skilled in the art. Examples of such methods and techniques include, without limitation, chelate dialysis, extrusion (with or without freeze-thaw), French press, homogenization, microemulsification, reverse phase evaporation, simple freeze-thaw, solvent dialysis, solvent infusion, solvent vaporization, sonication, and spontaneous formation. Preparation of the liposomes may be carried out in a solution, such as an aqueous saline solution, aqueous phosphate buffer solution, or sterile water. Liposome compositions also may be prepared by various processes involving shaking or vortexing. CD81 protein may be incorporated into the layers of a liposome such that its intracellular domain extends outside the liposome, and its extracellular domain extends into the interior of the liposome. The liposome containing CD81 then may be fused with an astrocyte, in accordance with known methods of fusion of liposomes to cell membranes, such that the CD81 protein is delivered into the membrane of the astrocyte with its intracellular domain extending into the interior of the astrocyte, and its extracellular domain extending outside the membrane of the astrocyte.

In the method of the present invention, CD81 also may be contacted with astrocytes, either in vitro or in vivo in a subject, by introducing into a sufficient number of astrocytes of the subject a nucleic acid encoding CD81, in a manner permitting expression of CD81. The nucleic acid may be introduced using conventional procedures known in the art, including, without limitation, electroporation, DEAE Dextran transfection, calcium phosphate transfection, monocationic liposome fusion, polycationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, and naked DNA transfer, or any combination thereof. Recombinant viral vectors suitable for gene therapy include, but are not limited to, vectors derived from the genomes of viruses such as retrovirus, HSV, adenovirus, adeno-associated virus, Semiliki Forest virus, cytomegalovirus, and vaccinia virus. The amount of nucleic acid encoding CD81 to be used is an amount that will express CD81 protein in an amount effective to inhibit proliferation of astrocytes, as defined above. These amounts may be readily determined by the skilled artisan.

It is also within the confines of the present invention that a nucleic acid encoding CD81 may be introduced into suitable cells in vitro, using conventional procedures, to achieve expression in the cells of CD81 protein. Cells expressing CD81 protein then may be introduced into a subject to inhibit proliferation of astrocytes in vivo. In such ex vivo gene therapy approaches, the cells are preferably removed from the subject, subjected to DNA techniques to incorporate nucleic acid encoding CD81, and then reintroduced into the subject.

The ability of CD81 to modulate astrocyte proliferation renders CD81 particularly useful for treating conditions associated with a defect in astrocyte proliferation. As used herein, “a defect in astrocyte proliferation” includes pathologic proliferation of astrocytes in a particular tissue, as compared with normal proliferation in the same type of tissue. It is believed that, by modulating astrocyte proliferation, CD81 will be useful for the treatment of conditions associated with defects in astrocyte proliferation. It is further believed that CD81 would be effective either alone or in combination with therapeutic agents, such as chemotherapeutic agents or antiviral agents, which are typically used in the treatment of these conditions.

Accordingly, the present invention provides a method for treating a condition associated with a defect in astrocyte proliferation in a subject in need of treatment, comprising contacting astrocytes in the subject with an amount of CD81 effective to inhibit proliferation of astrocytes, thereby treating the condition. As described above, the subject may be any animal, but is preferably a mammal (e.g., humans, domestic animals, and commercial animals). More preferably, the subject is a human.

Examples of conditions associated with a defect in astrocyte proliferation include, without limitation, astrocytosis, glial scars, hyperplasia, neoplasia, and neuritic plaques (particularly those commonly found in Alzheimer's disease patients). As used herein, “astrocytosis” refers to the proliferation of astrocytes owing to a destruction of nearby neurons. As further used herein, “hyperplasia” refers to the abnormal multiplication or increase in the number of normal astrocytes, in normal arrangement, within a tissue. In one embodiment of the present invention, the condition associated with a defect in astrocyte proliferation is astrocytosis. In another embodiment of the present invention, the condition associated with a defect in astrocyte proliferation is a neuritic plaque.

Astrocytosis, glial scars, hyperplasia, neoplasia, neuritic plaques, and other conditions associated with a defect in astrocyte proliferation may be caused by, or associated with, a variety of factors, including, without limitation, neuronal cell death and neural degeneration resulting from neurodegenerative diseases, CNS traumas, and the acquired secondary effects of non-neural dysfunction. Examples of neurodegenerative diseases include, without limitation, Alzheimer's disease, amyotrophic lateral sclerosis (Lou Gehrig's Disease), Binswanger's disease, Huntington's chorea, multiple sclerosis, myasthenia gravis, Parkinson's disease, and Pick's disease. Examples of CNS traumas include, without limitation, blunt trauma, hypoxia, and invasive trauma. Examples of acquired secondary effects of non-neural dysfunction include, without limitation, cerebral palsy, congenital hydrocephalus, muscular dystrophy, stroke, and vascular dementia.

In the treatment of a condition associated with a defect in astrocyte proliferation, CD81 may be contacted with astrocytes by introducing the CD81 protein into the membranes of astrocytes, in accordance with known methods (e.g., liposome delivery), as described above. The amount of CD81 protein to be used is an amount effective to inhibit proliferation of astrocytes, as defined above, and may be readily determined by the skilled artisan.

Alternatively, in accordance with known methods, including those described above, CD81 may be contacted with astrocytes to treat a condition associated with a defect in astrocyte proliferation by introducing into the astrocytes a nucleic acid encoding CD81, in a manner permitting expression of CD81 protein. The nucleic acid may be introduced using conventional procedures known in the art, including, without limitation, electroporation, DEAE Dextran transfection, calcium phosphate transfection, monocationic liposome fusion, polycationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, and naked DNA transfer, or any combination thereof. Recombinant viral vectors suitable for gene therapy include, but are not limited to, vectors derived from the genomes of viruses such as retrovirus, HSV, adenovirus, adeno-associated virus, Semiliki Forest virus, cytomegalovirus, and vaccinia virus. The amount of nucleic acid encoding CD81 to be used is an amount that will express CD81 protein in an amount effective to inhibit proliferation of astrocytes, as defined above. These amounts may be readily determined by the skilled artisan.

The present invention is also directed to a method for inhibiting proliferation of astrocytic tumor cells, by contacting astrocytic tumor cells with an amount of CD81 effective to inhibit proliferation of astrocytic tumor cells. As used herein, the term “astrocytic tumor cells” refers to a tumorigenic form of astrocytes (i.e., transformed astrocytes), and includes astrocytoma cells (i.e., cells of all astrocytomas, including, without limitation, Grades I-IV astrocytomas, anaplastic astrocytoma, astroblastoma, astrocytoma fibrillare, astrocytoma protoplasmaticum, gemistocytic astrocytoma, and glioblastoma multiforme). As defined above, “CD81” includes a CD81 protein (p27), a CD81 analogue, and a CD81 derivative.

The method of the present invention may be used to inhibit proliferation of astrocytic tumor cells in vitro, or in vivo in a subject. As used herein, the term “inhibit proliferation of astrocytic tumor cells” means inhibit cell division and growth of astrocytic tumor cells, and includes limiting the proliferative rate of astrocytic tumor cells. Inhibition of the growth and proliferation of astrocytic tumor cells may be detected by known procedures, including any of the methods, molecular procedures, and assays disclosed herein.

In accordance with the methods of the present invention, CD81 may be contacted with astrocytic tumor cells in vitro, or in vivo in a subject, by introducing the CD81 protein into the membranes of astrocytic tumor cells, or by introducing into the astrocytic tumor cells a nucleic acid encoding CD81 in a manner permitting expression of CD81 protein. The subject may be any animal, but is preferably a mammal (e.g., humans, domestic animals, and commercial animals). More preferably, the subject is a human. The astrocytic tumor cells may be found in neural tissue and other tissue of the nervous system of the subject, either alone or with other types of cells, including, without limitation, neurons and oligodendroglia. Astrocytic tumor cells may be detected in tissue of the subject by standard detection methods readily determined from the known art, including, without limitation, immunological techniques (e.g., immunohistochemical staining), fluorescence imaging techniques, and microscopic techniques.

CD81 protein may be introduced into the membranes of astrocytic tumor cells, either in vitro or in vivo in a subject, by known techniques used for the introduction of proteins (e.g., liposome delivery), as described above. For liposome delivery, liposomal vesicles and liposome compositions may be prepared using a variety of conventional techniques, including those described above. CD81 protein may be incorporated into the layers of a liposome such that its extracellular domain extends outside the liposome, and its intracellular domain extends into the interior of the liposome. The liposome containing CD81 then may be fused with astrocytic tumor cells, in accordance with known methods of fusion of liposomes to cell membranes, such that the CD81 protein is delivered into the membrane of the astrocytic tumor cells. The amount of CD81 protein to be used is an amount effective to inhibit proliferation of astrocytic tumor cells, as defined above, and may be readily determined by the skilled artisan.

In the method of the present invention, CD81 also may be contacted with astrocytic tumor cells, either in vitro or in vivo in a subject, by introducing into a sufficient number of astrocytic tumor cells of the subject a nucleic acid encoding CD81, in a manner permitting expression of CD81. The nucleic acid may be introduced using conventional procedures known in the art, including in vivo gene therapy, ex vivo gene therapy, and all other above-described procedures. Recombinant viral vectors suitable for gene therapy include all of the vectors described above. The amount of nucleic acid encoding CD81 to be used is an amount that will express CD81 protein in an amount effective to inhibit proliferation of astrocytic tumor cells, as defined above. These amounts may be readily determined by the skilled artisan.

The ability of CD81 to modulate astrocyte proliferation, and the absence of CD81 from astrocytic tumor cell lines, together suggest that CD81 may be useful for treating astrocytomas and other conditions associated with proliferation of astrocytic tumor cells. Furthermore, it is believed that CD81 would be effective either alone or in combination with therapeutic agents, such as chemotherapeutic agents or antiviral agents, which are typically used in the treatment of these conditions.

Accordingly, the present invention provides a method for treating a condition associated with proliferation of astrocytic tumor cells in a subject in need of treatment, comprising contacting astrocytic tumor cells in the subject with an amount of CD81 effective to inhibit proliferation of astrocytic tumor cells, thereby treating the condition. As described above, the subject may be any animal, but is preferably a mammal (e.g., humans, domestic animals, and commercial animals). More preferably, the subject is a human.

As used herein, the term “conditions associated with proliferation of astrocytic tumor cells” includes pathologic proliferation of astrocytic tumor cells, such as astrocytoma cells, and other forms of neoplasia. The term “neoplasia”, as further used herein, refers to the uncontrolled and progressive multiplication of astrocytic tumor cells under conditions that would not elicit, or would cause cessation of, multiplication of normal astrocytes. Neoplasia results in the formation of a “neoplasm”, which is defined herein to mean any new and abnormal growth, particularly a new growth of tissue, in which the growth of cells is uncontrolled and progressive. Neoplasms include benign tumors and malignant tumors (e.g., astrocytomas, such as Grades I-IV astrocytomas, anaplastic astrocytoma, astroblastoma, astrocytoma fibrillare, astrocytoma protoplasmaticum, gemistocytic astrocytoma, and glioblastoma multiforme, and other brain tumors). Malignant neoplasms are distinguished from benign in that the former show a greater degree of anaplasia, or loss of differentiation and orientation of cells, and have the properties of invasion and metastasis. Thus, neoplasia includes “cancer”, which herein refers to a proliferation of astrocytic tumor cells having the unique trait of loss of normal controls, resulting in unregulated growth, lack of differentiation, local tissue invasion, and metastasis. In one embodiment of the present invention, the condition associated with proliferation of astrocytic tumor cells is an astrocytoma.

In the treatment of a condition associated with proliferation of astrocytic tumor cells, CD81 may be contacted with astrocytic tumor cells by introducing the CD81 protein into the membranes of astrocytic tumor cells, in accordance with known methods (e.g., liposome delivery), as described above. The amount of CD81 protein to be used is an amount effective to inhibit proliferation of astrocytic tumor cells, as defined above, and may be readily determined by the skilled artisan.

Alternatively, in accordance with known methods, including those described above, CD81 may be contacted with astrocytic tumor cells to treat a condition associated with a defect in astrocytic tumor cell proliferation by introducing into the astrocytic tumor cells a nucleic acid encoding CD81, in a manner permitting expression of CD81 protein. The nucleic acid may be introduced using conventional procedures known in the art, including in vivo gene therapy, ex vivo gene therapy, and all above-described procedures. Recombinant viral vectors suitable for gene therapy include all vectors described above. The amount of nucleic acid encoding CD81 to be used is an amount that will express CD81 protein in an amount effective to inhibit proliferation of astrocytic tumor cells, as defined above. These amounts may be readily determined by the skilled artisan.

The present invention further provides a method for inhibiting proliferation of astrocytes, comprising contacting astrocytes with a modulator of CD81 expression, in an amount effective to inhibit proliferation of astrocytes. The modulator may be a protein, polypeptide, peptide, nucleic acid (including DNA or RNA), antibody, Fab fragment, F(ab′)₂ fragment, molecule, compound, antibiotic, drug, neuron, or other agent, as defined herein, that induces or upregulates CD81 expression. Examples of modulators include, without limitation, neurons, FK506, and other neuroimmunophilins.

Additional modulators of CD81 may be identified using a simple screening assay based on procedures described below. For example, to screen for candidate modulators of CD81, astrocytic tumor cells may be plated onto microtiter plates, then contacted with a library of drugs. Any resulting expression of CD81 may be detected using nucleic acid hybridization and/or immunological techniques known in the art, including an ELISA. Modulators of CD81 will be those drugs which induce or upregulate expression of CD81. In this manner, agents also may be screened for their ability to inhibit proliferation of astrocytes or astrocytic tumor cells using CD81 expression as an indicator that cell division or growth of astrocytes or astrocytic tumor cells is decreasing in rate, or has stopped.

The present invention further provides a method for inhibiting proliferation of astrocytic tumor cells, comprising contacting astrocytic tumor cells with a modulator of CD81 expression, in an amount effective to inhibit proliferation of astrocytic tumor cells. Examples of such modulators of CD81 expression include all of those described above. Additional modulators of CD81 may be screened in accordance with the above-described methods.

The present invention also provides a method for treating a condition associated with a defect in astrocyte proliferation in a subject in need of treatment, by administering to the subject an amount of CD81 effective to treat the condition associated with a defect in cell proliferation. The subject may be any animal, but is preferably a mammal (e.g., humans, domestic animals, and commercial animals). More preferably, the subject is a human.

As described above, examples of conditions associated with a defect in astrocyte proliferation include, without limitation, astrocytosis, glial scars, hyperplasia, neoplasia, and neuritic plaques (particularly those commonly found in Alzheimer's disease patients). Additionally, conditions associated with a defect in astrocyte proliferation may be caused by, or associated with, a variety of factors, including, without limitation, neuronal cell death and neural degeneration resulting from neurodegenerative diseases, CNS traumas, and the acquired secondary effects of non-neural dysfunction. Examples of neurodegenerative diseases, CNS traumas, and acquired secondary effects of non-neural dysfunction include all of those described above. In one embodiment of the present invention, the condition associated with a defect in astrocyte proliferation is astrocytosis.

The CD81 of the present invention is administered to a subject in need of treatment for a condition associated with a defect in astrocyte proliferation in an amount that is effective to treat the condition associated with a defect in astrocyte proliferation in the subject. As used herein, the phrase “effective to treat the condition associated with a defect in astrocyte proliferation” means effective to ameliorate or minimize the clinical impairment or symptoms of the condition associated with a defect in astrocyte proliferation. For example, where the condition associated with a defect in astrocyte proliferation is astrocytosis, the clinical impairment or symptoms of the astrocytosis may be ameliorated or minimized by reducing the mass of astrocytes produced by the astrocytosis, thereby minimizing any potential obstruction of axons which may occur. The amount of CD81 effective to treat a condition associated with a defect in astrocyte proliferation in a subject in need of treatment therefor will vary depending upon the particular factors of each case, including the type of defect in astrocyte proliferation, the stage of the defect in astrocyte proliferation, the subject's weight, the severity of the subject's condition, and the method of administration. This amount can be readily determined by the skilled artisan.

According to the method of the present invention, CD81 may be administered to a human or animal subject by known procedures, including, without limitation, oral administration, parenteral administration, transdermal administration, and administration through an osmotic mini-pump. Preferably, the CD81 is administered parenterally, by intracranial, intraspinal, intrathecal, or subcutaneous injection. The CD81 of the present invention also may be administered to a subject in accordance with any of the above-described methods for effecting in vivo contact between astrocytes and CD81.

For oral administration, the formulation of CD81 may be presented as capsules, tablets, powders, granules, or as a suspension. The formulation may have conventional additives, such as lactose, mannitol, corn starch, or potato starch. The formulation also may be presented with binders, such as crystalline cellulose, cellulose derivatives, acacia, corn starch, or gelatins. Additionally, the formulation may be presented with disintegrators, such as corn starch, potato starch, or sodium carboxymethylcellulose. The formulation also may be presented with dibasic calcium phosphate anhydrous or sodium starch glycolate. Finally, the formulation may be presented with lubricants, such as talc or magnesium stearate.

For parenteral administration (i.e., administration by injection through a route other than the alimentary canal), CD81 may be combined with a sterile aqueous solution that is preferably isotonic with the blood of the subject. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulations may be presented in unit or multi-dose containers, such as sealed ampoules or vials. The formulation may be delivered by any mode of injection, including, without limitation, epifascial, intracapsular, intracranial, intracutaneous, intrathecal, intramuscular, intraorbital, intraperitoneal, intraspinal, intrasternal, intravascular, intravenous, parenchymatous, or subcutaneous.

For transdermal administration, CD81 may be combined with skin penetration enhancers, such as propylene glycol, polyethylene glycol, isopropanol, ethanol, oleic acid, N-methylpyrrolidone, and the like, which increase the permeability of the skin to the CD81, and permit the CD81 to penetrate through the skin and into the bloodstream. The CD81/enhancer compositions also may be further combined with a polymeric substance, such as ethylcellulose, hydroxypropyl cellulose, ethylene/vinylacetate, polyvinyl pyrrolidone, and the like, to provide the composition in gel form, which may be dissolved in solvent, such as methylene chloride, evaporated to the desired viscosity, and then applied to backing material to provide a patch. CD81 may be administered transdermally at the site in the subject where neural trauma has occurred, or where the defect in astrocyte proliferation is localized. Alternatively, CD81 may be administered transdermally at a site other than the affected area, in order to achieve systemic administration.

The CD81 of the present invention also may be released or delivered from an osmotic mini-pump or other time-release device. The release rate from an elementary osmotic mini-pump may be modulated with a microporous, fast-response gel disposed in the release orifice. An osmotic mini-pump would be useful for controlling release, or targeting delivery, of CD81.

The present invention also provides a method for treating a condition associated with proliferation of astrocytic tumor cells in a subject in need of treatment therefor, by administering to the subject an amount of CD81 effective to treat the condition associated with proliferation of astrocytic tumor cells. The subject may be any animal, but is preferably a mammal (e.g., humans, domestic animals, and commercial animals). More preferably, the subject is a human. As described above, examples of conditions associated with proliferation of astrocytic tumor cells include, without limitation, astrocytomas, brain tumors, and other forms of neoplasia. In one embodiment of the present invention, the condition associated with proliferation of astrocytic tumor cells is an astrocytoma.

The CD81 of the present invention is administered to a subject in need of treatment for a condition associated with proliferation of astrocytic tumor cells in an amount that is effective to treat the condition associated with proliferation of astrocytic tumor cells in the subject. As used herein, the phrase “effective to treat the condition associated with proliferation of astrocytic tumor cells” means effective to ameliorate or minimize the clinical impairment or symptoms of the condition associated with proliferation of astrocytic tumor cells.

For example, where the condition associated with proliferation of astrocytic tumor cells is an astrocytoma, the clinical impairment or symptoms of the astrocytoma may be ameliorated or minimized by diminishing any pain or discomfort suffered by the subject; by extending the survival of the subject beyond that which would otherwise be expected in the absence of such treatment; by inhibiting or preventing the development or spread of the neoplasm; or by limiting, suspending, terminating, or otherwise controlling the maturation and proliferation of astrocytic tumor cells in the astrocytoma. The amount of CD81 effective to treat a condition associated with proliferation of astrocytic tumor cells in a subject in need of treatment will vary depending upon the particular factors of each case, including the type of condition associated with proliferation of astrocytic tumor cells, the stage of the condition associated with proliferation of astrocytic tumor cells, the subject's weight, the severity of the subject's condition, and the method of administration. This amount can be readily determined by the skilled artisan.

According to the method of the present invention, CD81 may be administered to a human or animal subject by known procedures, including, without limitation, oral administration, parenteral administration, transdermal administration, and administration through an osmotic mini-pump. Preferably, the CD81 is administered parenterally, by intracranial, intraspinal, intrathecal, or subcutaneous injection. The CD81 of the present invention also may be administered to a subject in accordance with any of the above-described methods for effecting in vivo contact between astrocytic tumor cells and CD81.

For oral administration, the formulation of CD81 may be presented as capsules, tablets, powders, granules, as a suspension, or in any of the above-described formulations. For parenteral administration, CD81 may be combined with a sterile aqueous solution which is preferably isotonic with the blood of the subject. Such a formulation may be prepared in accordance with the above-described method of preparation. The formulations for parenteral administration may be presented in unit or multi-dose containers, such as sealed ampoules or vials, and may be delivered by any of the modes of injection described above.

For transdermal administration, CD81 may be combined with skin penetration enhancers, such as those described above. The CD81/enhancer compositions also may be further combined with a polymeric substance, such as any of those described above, to provide the composition in gel form. CD81 may be administered transdermally at the site in the subject where astrocytic tumor cell proliferation has occurred. Alternatively, CD81 may be administered transdermally at a site other than the affected area, in order to achieve systemic administration. Finally, the CD81 of the present invention also may be released or delivered from an osmotic mini-pump or other time-release device, as described above.

The present invention further provides a method for treating a condition associated with a defect in astrocyte proliferation in a subject in need of treatment, by administering to the subject a modulator of CD81 expression in an amount effective to induce or enhance expression of CD81 and treat a condition associated with a defect in astrocyte proliferation, as defined above, in the subject. Examples of such modulators of CD81 expression include all of those described above. Additional modulators of CD81 may be screened in accordance with the above-described methods. The modulator of CD81 may be administered to a subject in any of the formulations, and by any of the modes of administration, described above.

The present invention also provides a method for treating a condition associated with proliferation of astrocytic tumor cells in a subject in need of treatment, by administering to the subject a modulator of CD81 expression in an amount effective to induce or enhance expression of CD81 and treat the condition associated with proliferation of astrocytic tumor cells, as defined above, in the subject. Examples of such modulators of CD81 expression include all of those described above. Additional modulators of CD81 may be screened in accordance with the above-described methods. The modulator of CD81 may be administered to a subject in any of the formulations, and by any of the modes of administration, described herein. Moreover, the modulator of CD81 also may be administered along with a chemotherapeutic agent, such as a ricin-conjugated CD81-binding protein.

In view of the foregoing, it is predicted that administration of CD81 will provide an effective treatment option for conditions associated with either a defect in astrocyte proliferation or a proliferation of astrocytic tumor cells. The therapies described herein offer real treatment options for inhibiting astrocyte and astrocytoma proliferation, without the massive side-effects and bystander effects that typically accompany the current treatment regimes. The population at risk for these conditions is large, and the needs currently are not being met.

The present invention further provides a pharmaceutical composition, comprising CD81 and a pharmaceutically-acceptable carrier, wherein CD81 is present in an amount sufficient or effective to treat a condition associated with a defect in astrocyte proliferation, as defined above, in a subject to whom said pharmaceutical composition is administered. Such a pharmaceutical composition would be useful for administering CD81 to a subject in need of treatment for a condition associated with a defect in astrocyte proliferation, in order to treat said condition. The CD81 is provided to the subject in an amount that is effective to treat the condition associated with a defect in astrocyte proliferation, as defined above, in the subject. This amount may be readily determined by the skilled artisan. The pharmaceutical composition may be administered to a subject in accordance with any of the methods of administration described above.

Formulations of the pharmaceutical composition of the present invention may be conveniently presented in unit dosage, and may be presented in oral dosage form (e.g., CD81 and a pharmaceutically-acceptable carrier may be combined in an ampule, capsule, pill, powder, or tablet) or in a form suitable for injection. The pharmaceutically-acceptable carrier may be a solid, liquid, or gel. Furthermore, the pharmaceutically-acceptable carrier of the present invention must be “acceptable” in the sense of being compatible with the other ingredients of the composition, and not deleterious to the recipient thereof. Examples of acceptable pharmaceutical carriers include carboxymethylcellulose, crystalline cellulose, glycerin, gum arabic, lactose, magnesium stearate, methyl cellulose, polypeptides, powders, saline, sodium alginate, starch, sucrose, talc, and water, among others. The carrier selected will depend upon the route of administration, and the form in which CD81 is introduced.

The formulations of the present invention may be prepared by methods well known in the pharmaceutical art. For example, CD81 may be brought into association with a carrier or diluent, as an emulsion, suspension, or solution. Moreover, CD81 may be blended, at need, with another component, to the extent that such blending does not impair the object of the present invention. Such other component may be suitably selected in accordance with the purpose of use and type of formulation. Optionally, one or more accessory ingredients (e.g., buffers, colorants, flavoring agents, surface active agents, and the like) also may be added.

The present invention also discloses a pharmaceutical composition, comprising nucleic acid encoding CD81 and a pharmaceutically-acceptable carrier, wherein the nucleic acid expresses CD81 in an amount sufficient or effective to treat a condition associated with a defect in astrocyte proliferation, as defined above, in a subject to whom said pharmaceutical composition is administered. Such a pharmaceutical composition would be useful for administering CD81 to a subject in need of treatment for a condition associated with a defect in astrocyte proliferation, in order to treat said condition in the subject. The nucleic acid is provided to the subject in an amount such that it expresses CD81 protein in an amount that is effective to treat a condition associated with a defect in astrocyte proliferation, as defined above, in the subject. These amounts may be readily determined by the skilled artisan. Additionally, the pharmaceutical composition may be administered to a subject in accordance with any of the above-described methods of administration and introduction of nucleic acids.

Formulations of the pharmaceutical composition of the present invention may be conveniently presented in unit dosage, and may be presented in a form suitable for administration of nucleic acid (e.g., by injection). The nucleic acid encoding CD81 may be presented in any form well known in the art for introduction of nucleic acids, including, without limitation, naked DNA, plasmid DNA, and vector DNA (including viral vectors, as described above), and may be prepared in accordance with methods well known in the arts of gene therapy and molecular genetics. In addition, the pharmaceutically-acceptable carrier of the present invention must be “acceptable” in the sense of being compatible with the other ingredients of the composition, and not deleterious to the recipient thereof. Examples of acceptable pharmaceutical carriers include carboxymethylcellulose, crystalline cellulose, glycerin, gum arabic, lactose, magnesium stearate, methyl cellulose, polypeptides, powders, saline, sodium alginate, starch, sucrose, talc, and water, among others. The carrier selected will depend upon the route of administration, and the form in which nucleic acid encoding CD81 is introduced.

The formulations of the pharmaceutical composition of the present invention may be prepared by methods well known in the pharmaceutical art. For example, nucleic acid encoding CD81 may be brought into association with a carrier or diluent, as an emulsion, suspension, or solution. Moreover, nucleic acid encoding CD81 may be blended, at need, with another component, to the extent that such blending does not impair the object of the present invention. Such other component may be suitably selected in accordance with the purpose of use and type of formulation. Optionally, one or more accessory ingredients (e.g., buffers, colorants, surface active agents, and the like) also may be added.

The present invention further provides a pharmaceutical composition, comprising CD81 and a pharmaceutically-acceptable carrier, wherein CD81 is present in an amount sufficient or effective to treat a condition associated with proliferation of astrocytic tumor cells, as defined above, in a subject to whom said pharmaceutical composition is administered. Such a pharmaceutical composition would be useful for administering CD81 to a subject in need of treatment for a condition associated with proliferation of astrocytic tumor cells, in order to treat said condition in the subject. The CD81 is provided to the subject in an amount that is effective to treat the condition associated with proliferation of astrocytic tumor cells, as defined above, in the subject. This amount may be readily determined by the skilled artisan. The pharmaceutical composition may be administered to a subject in accordance with any of the methods of administration, and in any of the formulations, described above. The formulations of the present invention may be prepared in accordance with methods well known in the pharmaceutical art, including those described above.

The present invention also discloses a pharmaceutical composition, comprising nucleic acid encoding CD81 and a pharmaceutically-acceptable carrier, wherein the nucleic acid expresses CD81 in an amount sufficient or effective to treat a condition associated with proliferation of astrocytic tumor cells, as defined above, in a subject to whom said pharmaceutical composition is administered. Such a pharmaceutical composition would be useful for administering CD81 to a subject in need of treatment for a condition associated with proliferation of astrocytic tumor cells, in order to treat said condition in the subject. The nucleic acid is provided to the subject in an amount such that it expresses CD81 protein in an amount that is effective to treat a condition associated with proliferation of astrocytic tumor cell, as defined above, in the subject. These amounts may be readily determined by the skilled artisan. Additionally, the pharmaceutical composition may be administered to a subject in accordance with any of the above-described methods of administration and introduction of nucleic acids, and in any of the formulations described above. The formulations of the pharmaceutical composition of the present invention may be prepared in accordance with methods well known in the pharmaceutical art, including those described above.

The present invention further provides a method for determining whether a subject has an astrocytoma, comprising assaying for CD81 expression a diagnostic sample of cells of astrocytic lineage of the subject, wherein no detection of expression of CD81 in cells of astrocytic lineage of the subject is diagnostic of an astrocytoma. The subject may be any animal, but is preferably a mammal (e.g., humans, domestic animals, and commercial animals). More preferably, the subject is a human. As used herein, “CD81” includes CD81 protein, cDNA, and mRNA.

As used herein, “no detection of expression of CD81” means that CD81 is not present in astrocytic tumor cells of the subject at a detectable level. As further used herein, the term “cells of astrocytic lineage” includes astrocytes and astrocytic tumor cells, as defined above. It is also within the confines of the present invention to provide a method for confirming a diagnosis of astrocytoma in a subject, comprising assaying for CD81 expression a diagnostic sample of cells of astrocytic lineage of the subject, wherein no detection of expression of CD81 in cells of astrocytic lineage of the subject is diagnostic of an astrocytoma.

According to the method of the present invention, the diagnostic sample of cells of astrocytic lineage of the subject may be assayed for CD81 expression in vitro, or in vivo in a subject. In accordance with the present invention, where the assay is performed in vitro, a diagnostic sample of cells of astrocytic lineage, or tissue containing cells of astrocytic lineage, may be removed from the subject using standard procedures, including biopsy and aspiration. Preferably, the diagnostic sample of cells or tissue is removed using multidirectional fine-needle aspiration biopsy (FNAB). This method of removal is preferred, as it is less invasive than a standard biopsy. The diagnostic sample taken from the subject may be, for example, any tissue known to have an astrocytoma, any tissue suspected of having an astrocytoma, or any tissue believed not to have an astrocytoma.

Protein may be isolated and purified from the diagnostic sample of the present invention using standard methods known in the art, including, without limitation, extraction from a tissue (e.g., with a detergent that solubilizes the protein) where necessary, followed by affinity purification on a column, chromatography (e.g., FTLC and HPLC), immunoprecipitation (with an antibody to CD81), and precipitation (e.g., with isopropanol and a reagent such as Trizol). Isolation and purification of the protein may be followed by electrophoresis (e.g., on an SDS-polyacrylamide gel). Nucleic acid may be isolated from a diagnostic sample using standard techniques known to one of skill in the art.

In accordance with the method of the present invention, an astrocytoma in a subject may be diagnosed by assaying a diagnostic sample of the subject for expression of CD81. Because CD81 is generally expressed in cells of astrocytic lineage from healthy, nondiseased subjects (i.e., those who do not have an astrocytoma), no detection of CD81 expression in a diagnostic sample of cells of astrocytic lineage of a subject is diagnostic of an astrocytoma. As used herein, “expression” means the transcription of the CD81 gene into at least one mRNA transcript, or the translation of at least one mRNA into a CD81 protein, as defined above. Accordingly, a diagnostic sample may be assayed for CD81 expression by assaying for CD81 protein (as defined above), cDNA, or mRNA. The appropriate form of CD81 will be apparent based on the particular techniques discussed herein.

In the method of the present invention, a diagnostic sample of cells of astrocytic lineage a subject may be assayed for CD81 expression, and CD81 expression may be detected in a diagnostic sample, using assays and detection methods readily determined from the known art, including, without limitation, immunological techniques, hybridization analysis, fluorescence imaging techniques, and/or radiation detection. For example, astrocytes or cells that are removed from the subject using FNAB may be analyzed using immunocytofluorometry (FACS analysis). In another embodiment of the present invention, the diagnostic sample is assayed for expression of CD81 using Northern blot analysis of CD81 mRNA extracted from cells of astrocytic lineage.

According to the method of the present invention, a diagnostic sample of the subject may be assayed for CD81 expression using an agent reactive with CD81. As used herein, “reactive” means the agent has affinity for, binds to, or is directed against CD81. As further used herein, an “agent” shall include a protein, polypeptide, peptide, nucleic acid (including DNA or RNA), antibody, Fab fragment, F(ab′)₂ fragment, molecule, compound, antibiotic, drug, and any combinations thereof. Moreover, an agent reactive with CD81 may be either natural or synthetic. The agent may be in the form of an antibody, a Fab fragment, an F(ab′)₂ fragment, a peptide, a polypeptide, a protein, and any combinations thereof. A Fab fragment is a univalent, antigen-binding fragment of an antibody, which is produced by papain digestion. An F(ab′)₂ fragment is a divalent antigen-binding fragment of an antibody, which is produced by pepsin digestion. Preferably, the agent is a high-affinity antibody labeled with a detectable marker. Where the agent is an antibody, the absence of expression of CD81 may be detected from binding studies using one or more antibodies immunoreactive with CD81, along with standard immunological detection techniques, such as Western blotting.

As used herein, the antibody of the present invention may be polyclonal or monoclonal, and may be produced by techniques well known to those skilled in the art. Polyclonal antibody, for example, may be produced by immunizing a mouse, rabbit, or rat with purified CD81. Monoclonal antibody then may be produced by removing the spleen from the immunized mouse, and fusing the spleen cells with myeloma cells to form a hybridoma which, when grown in culture, will produce a monoclonal antibody. Monoclonal antibodies that are reactive with CD81 also may be obtained from Pharmingen (San Diego, Calif.) (e.g., mAb 2F7) and Boehringer (Mannheim, Germany) (e.g., mAbs Eat1 and Eat2).

The antibodies used herein may be labeled with a detectable marker. Labeling of the antibody may be accomplished using one of the variety of different chemiluminescent and radioactive labels known in the art. The detectable marker of the present invention may be, for example, a nonradioactive or fluorescent marker, such as biotin, fluorescein (FITC), acridine, cholesterol, or carboxy-X-rhodamine, which can be detected using fluorescence and other imaging techniques readily known in the art. Alternatively, the detectable marker may be a radioactive marker, including, for example, a radioisotope. The radioisotope may be any isotope that emits detectable radiation, such as ³⁵S, ³²P, or ³H. Radioactivity emitted by the radioisotope can be detected by techniques well known in the art. For example, gamma emission from the radioisotope may be detected using gamma imaging techniques, particularly scintigraphic imaging. Preferably, the agent of the present invention is a high-affinity antibody labeled with a detectable marker. The antibodies of the present invention also may be incorporated into kits that include an appropriate labeling system, buffers, and other necessary reagents for use in a variety of detection and diagnostic applications.

Where the agent of the present invention is an antibody reactive with CD81, a diagnostic sample taken from the subject may be purified by passage through an affinity column which contains CD81 antibody as a ligand attached to a solid support such as an insoluble organic polymer in the form of a bead, gel, or plate. The antibody attached to the solid support may be used in the form of a column. Examples of suitable solid supports include, without limitation, agarose, cellulose, dextran, polyacrylamide, polystyrene, sepharose, or other insoluble organic polymers. The CD81 antibody may be further attached to the solid support through a spacer molecule, if desired. Appropriate binding conditions (e.g., temperature, pH, and salt concentration) may be readily determined by the skilled artisan. In a preferred embodiment, the CD81 antibody is attached to a sepharose column, such as Sepharose 4B.

Where the agent is an antibody, a diagnostic sample of the subject may be assayed for CD81 expression using binding studies that utilize one or more antibodies immunoreactive with CD81, along with standard immunological detection techniques. For example, the CD81 protein eluted from the affinity column may be subjected to an ELISA assay, Western blot analysis, flow cytometry, or any other immunostaining method employing an antigen-antibody interaction. Preferably, the diagnostic sample is assayed for CD81 expression using Western blotting.

Alternatively, a diagnostic sample of cells of astrocytic lineage of a subject may be assayed for CD81 expression using hybridization analysis of nucleic acid extracted from a sample of cells of astrocytic lineage, or tissue containing cells of astrocytic lineage, taken from the subject. According to this method of the present invention, the hybridization analysis may be conducted using Northern blot analysis of mRNA. This method also may be conducted by performing a Southern blot analysis of DNA using one or more nucleic acid probes which hybridize to nucleic acid encoding CD81. The nucleic acid probes may be prepared by a variety of techniques known to those skilled in the art, including, without limitation, the following: restriction enzyme digestion of CD81 nucleic acid; and automated synthesis of oligonucleotides having sequences that correspond to selected portions of the nucleotide sequence of the CD81 nucleic acid, using commercially-available oligonucleotide synthesizers, such as the Applied Biosystems Model 392 DNA/RNA synthesizer.

The nucleic acid probes used in the present invention may be DNA or RNA, and may vary in length from about 8 nucleotides to the entire length of the CD81 nucleic acid. The CD81 nucleic acid used in the probes may be derived from mammalian CD81. The nucleotide sequences for both rat, mouse, and human CD81 are known (19). Using these sequences as probes, the skilled artisan could readily clone corresponding CD81 cDNA from other species. In addition, the nucleic acid probes of the present invention may be labeled with one or more detectable markers. Labeling of the nucleic acid probes may be accomplished using one of a number of methods known in the art (e.g., nick translation, end labeling, fill-in end labeling, polynucleotide kinase exchange reaction, random priming, or SP6 polymerase for riboprobe preparation), along with one of a variety of labels (e.g., radioactive labels, such as ³⁵S, ³²P, or ³H, or nonradioactive labels, such as biotin, fluorescein (FITC), acridine, cholesterol, or carboxy-X-rhodamine (ROX)). Combinations of two or more nucleic acid probes (or primers), corresponding to different or overlapping regions of the CD81 nucleic acid, also may be used to detect expression of CD81, using, for example, PCR or RT-PCR, and may be included in kits for use in a variety of detection and diagnostic applications.

It is contemplated that the diagnostic sample in the present invention frequently will be assayed for CD81 expression not by the subject, nor by his/her consulting physician, but by a laboratory technician or other clinician. Accordingly, the method of the present invention further comprises providing to a subject's consulting physician a report of the results obtained upon assaying a diagnostic sample of the subject for CD81 expression.

The present invention also provides a method for treating astrocytoma in a subject or patient. The subject may be any animal, but is preferably a mammal (e.g., humans, domestic animals, and commercial animals). More preferably, the subject is a human. The method of the present invention comprises the steps of: (a) diagnosing an astrocytoma in the subject or patient by detecting an absence of expression of CD81 in cells of astrocytic lineage of the subject or patient; and (b) treating the astrocytoma diagnosed in the subject or patient. The absence of expression of CD81 in cells of astrocytic lineage of the subject or patient may be detected by any of the methods described above. The astrocytoma diagnosed in the subject or patient may be treated by any method or combination of methods commonly used to treat astrocytoma, including, without limitation, surgery, radiotherapy, chemotherapy, immunotherapy, and systemic therapy. Preferably, however, an astrocytoma which is diagnosed in accordance with the method described herein is treated by administering CD81 to the subject or patient, as described above.

It is also within the confines of the present invention to use detected levels of CD81 expression as a clinical or pathologic staging tool, to determine which treatment options may be appropriate. In particular, detection of CD81 expression may be used to determine whether any of the treatment methods of the present invention is appropriate. Moreover, detected levels of CD81 expression may be used to grade brain tumors, particularly astrocytomas.

The present invention further provides a method for assessing the efficacy of astrocytoma therapy in a subject who has undergone or is undergoing treatment for astrocytoma. The subject may be any animal, but is preferably a mammal (e.g., humans, domestic animals, and commercial animals). More preferably, the subject is a human. The method of the present invention comprises assaying for CD81 expression a diagnostic sample of astrocytic tumor cells of the subject, wherein no detection of expression of CD81 in astrocytic tumor cells of the subject is indicative of unsuccessful astrocytoma therapy. The diagnostic sample may be any of those described above, and may be assayed for expression of CD81 either in vitro or in vivo in a subject. In addition, the diagnostic sample may be assayed for expression of CD81 using all of the various assays and methods of detection described above. This method of the present invention provides a means of monitoring the effectiveness of astrocytoma therapy by permitting the periodic assessment of levels of CD81 expression in astrocytic tumor cells of the subject.

According to the method of the present invention, a diagnostic sample of astrocytic tumor cells of a subject may be assayed, and levels of CD81 expression may be assessed, at any time following the initiation of therapy to treat an astrocytoma. For example, levels of CD81 expression may be assessed while the subject or patient is still undergoing treatment for the astrocytoma. Where expression of CD81 remains absent from astrocytic tumor cells of the subject, a physician may choose to continue with the astrocytoma treatment. Where levels of CD81 expression become detectable in astrocytic tumor cells of the subject, and then increase through successive assessments, it may be an indication that the astrocytoma treatment is working, and that treatment doses could be decreased or even ceased. Where levels of CD81 do not noticeably increase through successive assessments, it may be an indication that the astrocytoma treatment is not working, and that treatment doses could be increased. Where CD81 expression is eventually detected in astrocytic tumor cells of a subject or patient at a level expected for normal, non-diseased astrocytes, a physician may conclude that the astrocytoma treatment has been successful, and that such treatment may cease. It is also within the confines of the present invention to assess levels of CD81 expression following completion of the subject's or patient's astrocytoma treatment, in order to determine whether the astrocytoma has recurred in the subject or patient. Furthermore, it is within the confines of the present invention to use assessed levels of CD81 expression as a clinical or pathologic staging tool, to determine the extent of astrocytoma in the subject or patient, to determine appropriate treatment options, and to provide prognostic information.

The present invention is described in the following Experimental Details section, which is set forth to aid in the understanding of the invention, and should not be construed to limit in any way the scope of the invention as defined in the claims which follow thereafter.

### EXPERIMENTAL DETAILS

1. Introduction

The establishment and maintenance of the appropriate number and type of constituent cells in the central nervous system (CNS) of the mammal is a daunting problem. Not only do the correct numbers of cells end up in the correct locations: in the absence of trauma or disease, the total number of cells remains relatively constant throughout life. In the case of most neurons, which are incapable of dividing, this implies that there are mechanisms to provide continuous support. With astrocytes, however, the situation is more complex, as these cells are able to re-enter the cell cycle at virtually any point in their histories, and do so in response to trauma and disease (15). In spite of this proliferative ability, the number of astrocytes remains largely unchanged throughout life (23, 24, 25). It has been demonstrated that astrocytes can be maintained out of the cell cycle while they are in direct contact with the neuronal surface (11, 12, 29). It is important to determine how this mitotic quiescence is established and maintained, as there are major sequelae, both positive and negative, that result from astrocyte proliferation in the adult mammal.

In the case of CNS injury, resulting astrocytosis is thought to be a major contributor to the formation of a glial scar, which in turn may play an important role in blocking regenerating axons (6). While the isolation of damaged tissue is likely to be an important aspect in re-establishing the blood-brain barrier, this behavior implies that such an isolated area is permanently removed from the neural tissue available for regeneration. Further, because astrocytes do proliferate throughout life, albeit at very low levels (7), they are vulnerable to errors in DNA replication, as well as viral integration. This makes these cells susceptible to transformation over the lifetime of the mammal. Indeed, in those people who have been diagnosed with a brain tumor, the majority of tumors will be of astrocyte lineage. In view of the foregoing, it is clear that an understanding of the basic biology of neuron-glial interaction may provide insight into the means by which astrocyte growth control is both initially achieved and maintained throughout life. Such knowledge will likely yield further insight into methods for re-establishing ordered growth in transformed cells.

While the cell biology of neuron-astrocyte interaction has been amply described, the molecular correlates of this cell biology have been largely under-explored. In order to begin to define the nature of these interactions, the inventor has undertaken a series of differential gene screens to compare expression patterns in purified astrocyte cultures with those in astrocytes that were co-cultured with neurons. To avoid the problem of contaminating astrocyte RNA with RNA from the “effector” neurons, the inventor took advantage of his earlier observation that neuronal cell membranes are sufficient to drive astrocytes into quiescence (29).

From that observation, the inventor identified a number of genes the expression of which was unregulated by the neuron-stimulated astrocyte. The majority of genes that were identified in this assay system were known, and had well-documented expression patterns in non-neural tissues. Some of the genes in the screen were known to be expressed in the nervous system, but little was known of their biological significance. Among this latter class was a tetraspanin, CD81, the expression of which had been shown in astrocytes, and was known to be upregulated following neural trauma (8). However, the function of CD81 in trauma, and in homeostasis, was previously undefined.

Using a combination of antibody perturbation, biochemical competition, and gene knockout studies, the inventor has shown that CD81 is a critical modulator of astrocyte growth control. This observation is critical, as astrocytosis results from numerous neural traumas, and the resulting glial scar is believed to present a major barrier to productive neural regeneration. In addition, astrocytomas are the predominant single form of brain cancer, with a prevalence on the order of 17,000 cases per year. All astrocytoma cells tested herein failed to express CD81 message or protein, raising the possibility that CD81 plays an important role in astrocyte tumor formation and/or metastasis.

2. Materials and Methods

A. Animals

Pregnant Sprague-Dawley rats and C57BL/6 mice were obtained from Charles Rivers Laboratories. CD81 heterozygous mice were backcrossed greater than 10 generations into the C57BL/6 background. The generation of these mice has been previously described (17). Heterozygous animals were crossed, and offspring were born with the expected Mendelian frequency. However, an attenuated postnatal viability was observed in the CD81−/− animals. The genotypes of the progeny of these crosses was determined exactly as described (17). Notably, in earlier backcrosses, there was normal Mendelian distribution and normal survival in the homozygous null animals.

B. Tissue Culture-Primary Neural Cells

Primary cerebellar neurons and astrocytes were prepared as described (18). In brief, cerebella were dissected from rat or mouse pups at postnatal day 4 or 5, the meninges were stripped, and the remaining tissue was washed in Ca²⁺/Mg²⁺ free PBS (CMF-PBS). The tissue then was trypsinized, triturated through decreasing caliber needles in the presence of DNase, and pelleted. The cells were resuspended in CMF-PBS, and the single cell suspension was overlaid and separated on a Percoll step gradient (30/60%; Amersham Pharmacia), all as described (18). Following extensive washing to remove residual Percoll, the neuron- and astrocyte-enriched fractions were further enriched: differential adhesion removed contaminating astrocytes from the neuron preparation, and treatment with anti-Thy1 and complement-mediated cytolysis removed neurons and fibroblasts from the astrocyte-enriched fraction. All cells were cultured in D¹⁰, consisting of DMEM (Gibco) supplemented with 10% heat-inactivated fetal bovine serum (FBS; Gemini-Bio-Products, Inc.), 10% heat-inactivated horse serum (Gemini-Bio-Products, Inc.), 1% non-essential amino acids (Gibco), penicillin-streptomycin (Gibco; 20 U/ml), Fungizone (Gibco; 0.25 μg/ml), and glucose at 0.6% final concentration. The astrocyte cell suspensions were seeded at 2×10⁵ cells/well in 24-well plates (Costar) or 5×10⁴ cells/well in 8-well Lab-Tek tissue culture chambers (Nalge Nunc), which had been treated with 50 μg/ml poly-L-lysine (Sigma). Effector neurons were added at a ratio of 2 neurons per astrocyte.

C. Astrocytoma cell lines

Several astroglial cell lines (rat C6 and 9L, human A172 and U251MG, and mouse LN308 and LN18) were grown in 100-mm tissue culture dishes (Falcon Labware) in D¹⁰.

D. Antibodies

Rabbit anti-cow glial fibrillary acidic protein (GFAP) antibodies, as well as TRITC-conjugated swine anti-rabbit antibodies, were obtained from DAKO A/S (Copenhagen, Denmark). Mouse monoclonal antibodies (mAbs) to bromodeoxyuridine (BrdU) and conjugated to FITC were obtained from Boehringer (Mannheim, Germany). Hamster mAb 2F7 against CD81 (1) and FITC-conjugated mouse anti-hamster mAbs were obtained from Pharmingen (San Diego, Calif.). Hamster mAbs Eat1 and Eat2 react with distinct epitopes in CD81, and have been recently described (18). TuJ1 recognizes a neuron-specific βIII subunit of tubulin, and was the generous gift of Dr. Tony Frankfurter. Alexia red conjugated goat anti-mouse secondary antibody was purchased from Molecular Probes, and mouse mAb anti-GST was purchased from Sigma. Biotinylated goat anti-mouse antibody and the Vectastain ABC kit were purchased from Vector Labs.

E. Fusion Proteins

Full-length SCIP and the region of the mouse CD81 encoding the large extracellular loop (LEL) were both cloned into pGEX expression vectors (5), to generate GST fusion proteins. Clones were sequenced, and DH5α E. coli were transformed with the respective clones and IPTG induced. The resulting lysate was enriched on glutathione-agarose beads, and the protein concentration was assessed by BCA assay (Pierce). The integrity of the material was determined by gel electrophoresis and immunoblotting with antigen-specific antibodies and/or anti-GST antibodies, all by standard techniques.

F. Northern Blot Analysis

Total RNA was extracted from cultured cells, as described by Chomczynski and Sacchi (2). 20 μg of RNA from each sample were electrophoretically separated on denaturing agarose gels, and transferred to nylon membranes (Micron Separations Inc.). The membranes were probed overnight at 42° C. with random primed, [³²P]dCTP-labeled mouse CD81 cDNA, washed sequentially (three times for 15 min at 65° C. in 2×SSC, 0.1% SDS; twice for 10 min at 65° C. in 0.2×SSC, 0.1% SDS; and once for 5 min at room temperature in 2×SSC), dried, and exposed to X-ray film.

G. Immunoblotting

Cultured cerebellar astrocytes and astrocytes co-cultured with neurons C6 astroglioma were washed twice in ice-cold PBS, scraped from the dishes, pelleted, and resuspended in hypotonic disruption buffer (10 mM HEPES (pH 7.9), 10 mM NaCl, 0.1 mM EGTA, 0.1 mM EDTA, 0.5 mM phenylmethylsulfonylfluoride, 0.5 mg/ml leupeptin, 0.7 mg/ml pepstatin A, and 1 mg/ml aprotinin). The samples were incubated on ice for 15 min, after which NP-40 was added to a final concentration of 1%. The detergent-soluble and—insoluble portions were separated by centrifugation. Protein concentrations were determined for the membrane-containing detergent-soluble fraction using a BCA assay (Pierce). 50 mg of protein were separated on a 10% SDS-polyacrylamide gel, and the proteins then were transferred onto nitrocellulose using a semidry blotter. The efficiency of transfer was determined by amido black staining. The membrane was blocked in Buffer A, containing 5% milk and 1% Triton-X 100 in Tris buffered saline. The membrane was then probed with goat anti-CD81 antiserum, followed by peroxidase-conjugated donkey anti-goat secondary antibody. The reaction product was visualized by ECL.

H. Immunofluorescence

Forty-eight hours after plating, cell cultures were washed in PBS and fixed in 4% paraformaldehyde in PBS at 4° C. for 30 min. Nonspecific binding was blocked by incubation in 10% FBS/PBS for 30 min at room temperature. The blocking solution was removed, and primary antibodies (2F7, Eat1, or Eat2 diluted in PBS) were added at 37° C. for 1 h. The cultures were rinsed, incubated in mouse FITC-conjugated anti-hamster antibodies at room temperature for 30 min, washed, and mounted (Pro Long Antifade Kit, Molecular Probes). The same types of cultures were also double-labeled for GFAP and BrdU incorporation with FITC-conjugated anti-BrdU antibodies (Sigma) (see below). The cultures were washed in PBS, and fixed in 4% paraformaldehyde/PBS at 4° C. for 30 min, washed in PBS, and permeabilized in 0.5% Triton X-100/PBS at room temperature for 10 min. Nonspecific binding was blocked by incubation in 10% FBS/PBS at room temperature for 30 min, and GFAP was visualized with a TRITC-conjugated secondary antibody.

I. In vitro proliferation assays

Cultures were established, either under control conditions, or in the presence of fusion protein or increasing concentrations of one of the mAbs as described herein. Twenty-four hours later, 10 μM BrdU (Sigma) was added, and the cultures were continued for an additional 24 h. Subsequently, the cells were fixed, and astrocytes were identified by GFAP staining, as described herein. To visualize BrdU incorporation, the chromatin was denatured in 2 M HCl for 30 min, washed extensively in PBS, and incubated with FITC-anti-BrdU antibodies at room temperature for 1 h. Following incubation, the cells were washed extensively, stained with bis-benzamide (Sigma) to determine total cell numbers, washed again, and mounted in Anti-Fade. The level of astrocyte proliferation was determined by dividing the number of BrdU-positive astrocytes by the total number of GFAP-positive cells per microscopic area. All proliferation assays were repeated at least three times; 30 microscopic areas were examined from each experimental sample (600-700 cells/sample, for a minimum total of ˜2000 cells per experimental point). Statistical analysis of the data was performed using two-tailed, students' t-test.

3. Results

A. CD81 is expressed on the cell surface of the astrocyte

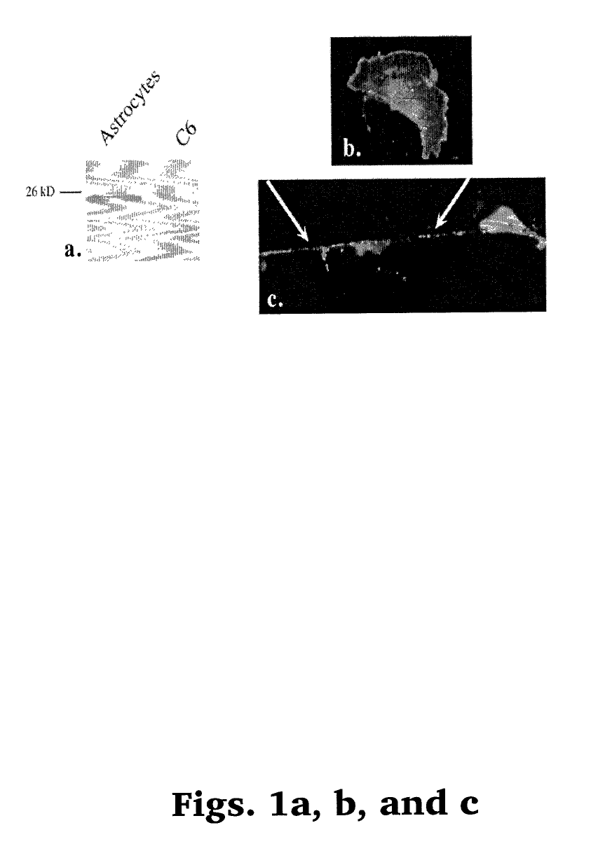

The inventor originally identified CD81 expression in astrocytes co-cultured with neuronal membranes by using a differential screening approach. To determine if CD81 protein was indeed expressed by astrocytes, the inventor established cultures of astrocytes or the astrocytoma cell line, C6, as previously described (28). Protein from 48-hour cultures was isolated, separated on an SDS-PAGE gel, and blotted with a polyclonal antibody against CD81. CD81 is constitutively expressed by cultured astrocytes, whereas the C6 glioma cells are CD81-negative (FIG. 1A). The addition of neurons to these cultures increased CD81 expression by 50-70% in astrocytes, but had no effect on the C6 cells (some data not shown). To localize CD81 expression on the astrocytes, an anti-CD81 monoclonal antibody (mAb), 2F7 (1), was used to stain rat astrocytes cultured alone, as well as co-cultures of astrocytes and neurons. FIG. 1B shows the punctate staining pattern of CD81 on the surface of astrocytes. When co-cultured with neurons, astrocytes extend complex processes that serve as guidance pathways for neuronal migration, and matrices for neuronal adhesion and differentiation (11, 29). Staining of neuron-astrocyte co-cultures showed a punctate pattern of CD81 expression, both on the astrocyte cell soma and along the processes (FIG. 1C, arrows). In contrast, neurons in these cultures failed to stain with the 2F7 antibody.

B. Anti-CD81 mAbs recognize unique, non-overlapping, extracellular epitopes

CD81 has four transmembrane domains, resulting in two loops in the extracellular domain. One is the small extracellular loop (SEL), and the other is the large extracellular loop (LEL). When the 2F7 antibody was used on live, non-permeabilized astrocytes, results indicated that it recognizes an extracellular epitope (FIGS. 1A and 1B). Recent work has shown that the 2F7 mAb recognizes a conformationally-dependent epitope that requires the presence of both the SEL and LEL of CD81. While Eat2 has a higher affinity for CD81 than does 2F7, it also requires both loops for antigen binding. In contrast, Eat1 recognizes an epitope within the LEL (18). In an effort to determine if any of these mAbs was able to block neuron-astrocyte interactions, the inventor established co-cultures in the presence of these reagents.

C. Neuron-astrocyte interactions are blocked in vitro by mAb Eat1

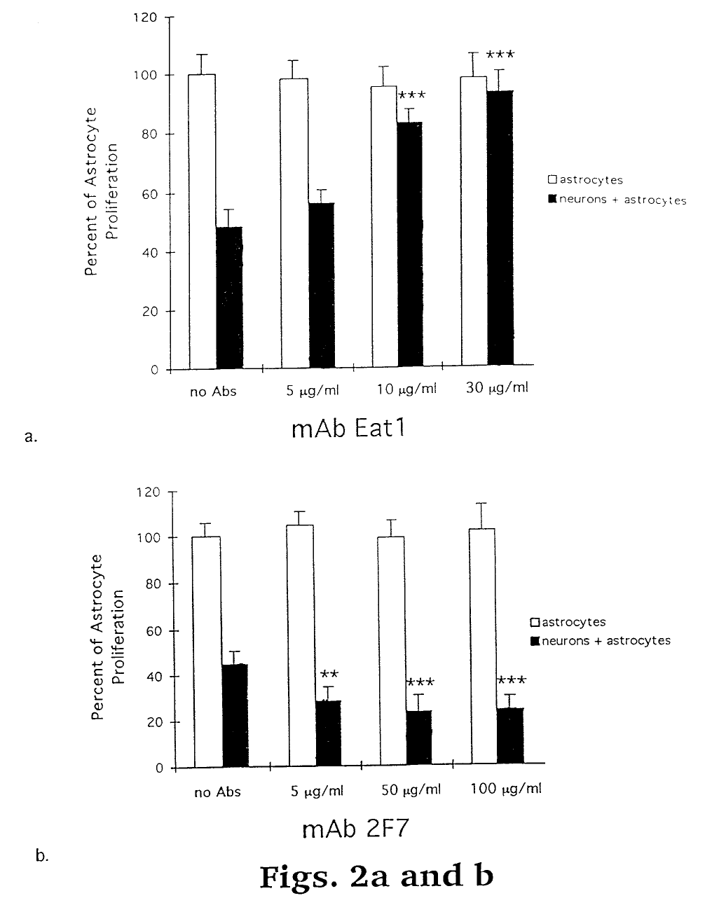

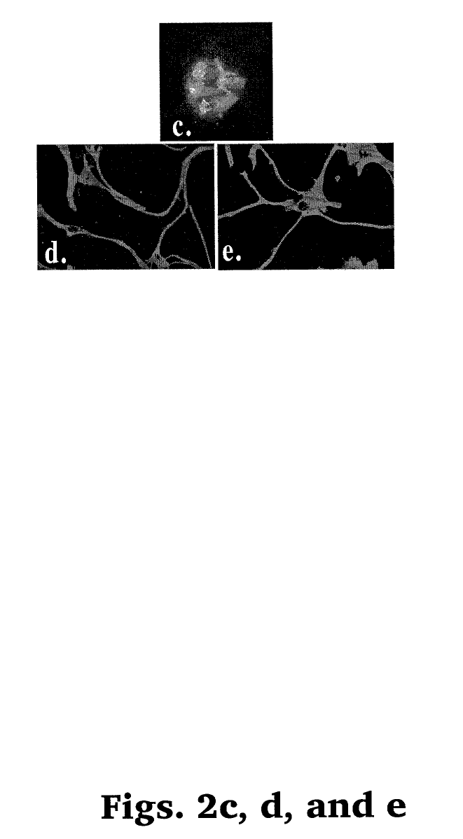

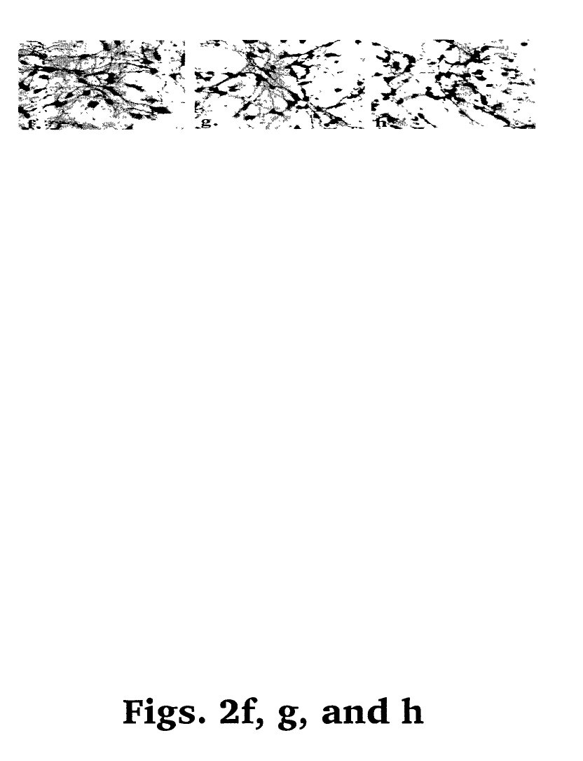

In order to determine the potential efficacy of Eat1 and Eat2 in blocking neuron-induced astrocyte differentiation and cell-cycle exit, the inventor established co-cultures of cerebellar granule cells and astrocytes. The cultures were allowed to grow for 48 h, with BrdU added in the last 24 h of culture. As can be seen in FIG. 2A, there is a loss of neuron-induced astrocyte growth arrest, which is dependent on the concentration of the Eat1 antibody. Notably, the neurons in this culture were viable, and adhered to the astrocytes and extended neurites (see below). Previous work has shown that cerebellar granule cells are exquisitely dependent upon astrocytes and astrocyte-derived factors for survival (13). The inventor's observation that the Eat1 mAb blocked neuron-dependent astrocyte proliferative arrest, but not trophic support, suggests that all normal neuron-astrocyte interactions are not lost under these conditions.

Eat2 had no apparent effect on neuron-astrocyte interactions: neuron-astrocyte co-cultures established in the presence of Eat2 were indistinguishable from control co-cultures. Under both control and Eat2 conditions, astrocytes withdrew from the cell cycle, and extended complex processes, when challenged with neurons (FIGS. 2D and 2E). In contrast, the addition of mAb 2F7 enhanced neuron-induced astrocyte proliferative arrest (FIG. 2B), suggesting that the addition of this antibody to the co-culture system augmented neuron-dependent astrocyte growth arrest. Taken with the Eat1 data, these observations show that alterations in CD81 bioavailability and/or conformation have a profound effect on modulating astrocytic proliferative responses to neurons.

It has been previously shown that astrocyte proliferation and process formation in response to neuronal contact are separable events. Contact with neuronal membranes is sufficient for astrocyte growth control, but viable neurons are required for both cell-cycle exit and process formation (29, 30). While the inventor saw no differences in astrocytic process outgrowth in neuron-astrocyte co-cultures under control conditions or in the presence of either mAb 2F7 or Eat2, there was a remarkable difference between those cultures and co-cultures established in the presence of Eat1. Examples of these stark differences can be seen in FIGS. 2C and 2D, in which neuron-astrocyte co-cultures were set up in the presence of Eat1 and Eat2, respectively. In the presence of Eat1 (FIG. 2C), the astrocytes failed to extend typical processes in response to neurons, and remained in the cell-cycle. The astrocytes depicted in FIG. 2C were imaged with anti-GFAP antiserum. These have the appearance of a cluster of daughter cells that arose in situ. In contrast, in the presence of Eat2 (FIG. 2D), the GFAP-expressing astrocytic processes are long, and of a complexity that is indistinguishable from astrocytic responses seen in co-cultures of astrocytes and viable, wild-type granule cell neurons (FIG. 2E). In addition, the integrity of neuritic processes is uncompromised in the presence of the anti-CD81 mAbs Eat1 and 2F7 (FIGS. 2F, 2G, and 2H). These data demonstrate that the effects of the anti-CD81 mAbs occur at the level of the astrocyte, and are not the result of a reduction in viability, or attenuation of axonogenic capabilities, of the neuron. Moreover, the data show that the treated astrocytes are able to support neuronal survival and axonal outgrowth, further highlighting the specificity of the role of CD81 in one aspect of astrocyte interactions with cognate neurons.

D. Soluble GST-CD81 fusion protein binds to the neuronal cell surface and competes for astrocyte-expressed CD81

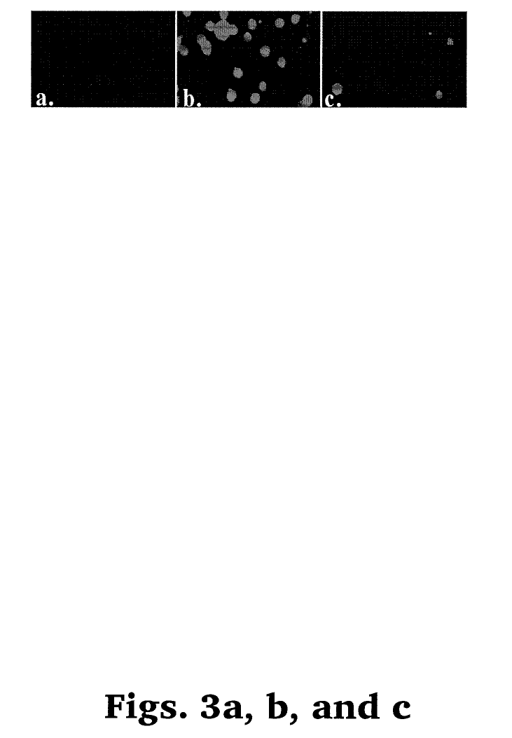

The antibody blocking studies were suggestive of a significant role of CD81 in mediating neuron-astrocyte interactions. However, as with any antibody blocking experiments, there is always a concern about steric inhibition. Therefore, to further extend these observations, the inventor used a soluble mouse GST-CD81 large extracellular loop (GST-CD81 (LEL)) fusion protein in an effort to compete for neuronal binding to the astrocyte. To determine if the fusion protein was able to bind to neurons, astrocytes, or both, the inventor isolated and purified the respective cell types, as described (28). The viable cells were incubated with 10 μg/ml of GST-CD81 (LEL) on ice for 1 h. Thereafter, the cells were fixed, then stained with an anti-GST antibody to avoid staining endogenous CD81. The GST-CD81 (LEL) fusion protein adhered to the neuronal fraction, but not the astrocytic fraction (FIG. 3). The few stained cells seen in the astrocyte-enriched fraction were probably neurons, based on the size of the cell somata, and the shape of the cells. Astrocytes in culture become flat, and bipolar or tripolar, unlike these cells.

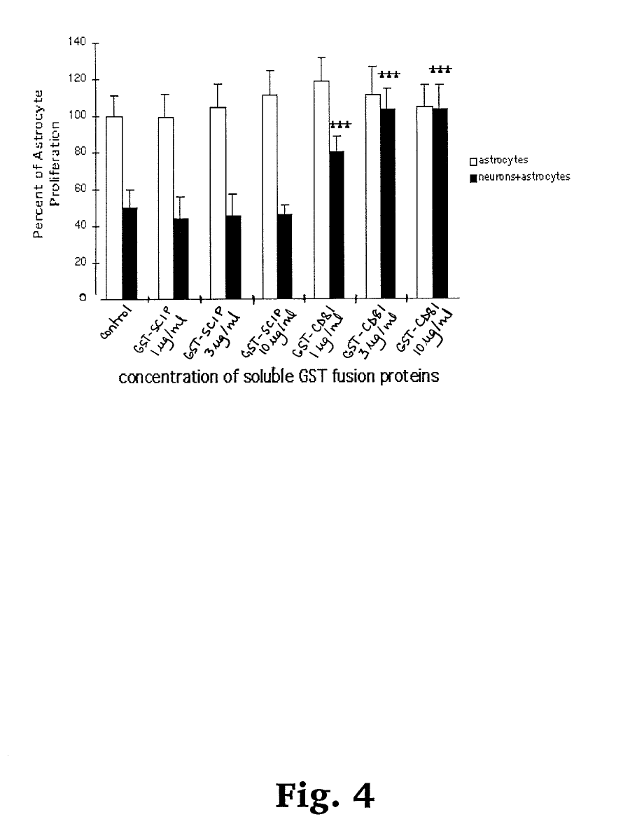

The observation that the CD81 (LEL) protein adhered to the surface of the neuron suggests the existence of a CD81 receptor on these cells. This putative receptor may be potentially involved in normal neuron-astrocyte interactions. To determine if blocking such a receptor would block the ability of the neuron to bind to CD81 and to differentiate the astrocyte via CD81, the inventor added increasing concentrations of either GST-CD81 (LEL) protein or an irrelevant GST fusion protein, GST-SCIP. The soluble CD81 blocked normal neuron-induced astrocyte proliferative arrest in a dose-dependent manner, while the GST-SCIP had no effect (FIG. 4). Neither fusion protein had any visible effects on neuronal survival or differentiation. Based on the neuronal binding patterns, as well as the blocking, these data suggest that the soluble CD81 is competing for receptors on the neuron, thereby blocking normal neuron-induced, CD81-mediated proliferative arrest (FIG. 4).

E. Astrocyte cell-cycle withdrawal is CD81-dependent

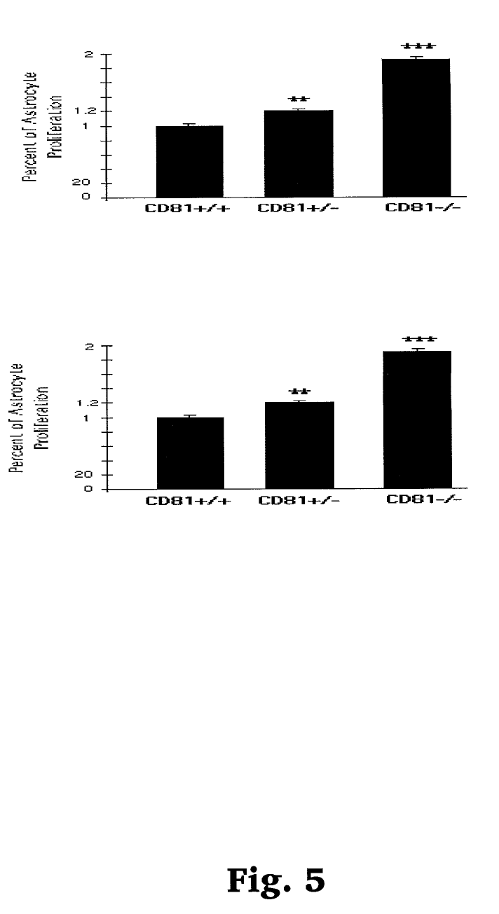

In addition to its expression on the astrocyte, CD81 is also expressed by numerous cell types, including lymphocytes. In the immune system, CD81 has been shown to play a vital role, as evidenced by the impaired immunity observed in CD81-deficient mice (16, 17, 20, 27). Heterozygotic CD81 mice were backcrossed 10 generations onto a C57BL/6 background, to establish the CD81 deletion in a pure C57BL/6 genotype. Mixed neuron-astrocyte cultures were established from animals immediately after birth. These CD81−/− animals were harvested in early postnatal life because they have severely decreased viability beyond the first hours of birth. At the time of harvesting, the additional neural tissue was taken for simultaneous genotyping. The cultures were established, and allowed to grow for 48 h. BrdU was added in the final 24 h of culture. The cells then were fixed and stained, and astrocyte proliferation was determined by BrdU and GFAP double labeling. The proliferation data was tabulated before the genotype of the respective cultures was unblinded. The extent of astrocyte proliferation in the wild-type co-culture was set at 1. With respect to this level of proliferation, CD81+/− animals showed a 20% increase in astrocyte BrdU incorporation, whereas the CD81−/− astrocytes showed a doubling of astrocyte proliferation (FIG. 5).

F. CD81 is absent in a variety of astrocytic tumor cell lines

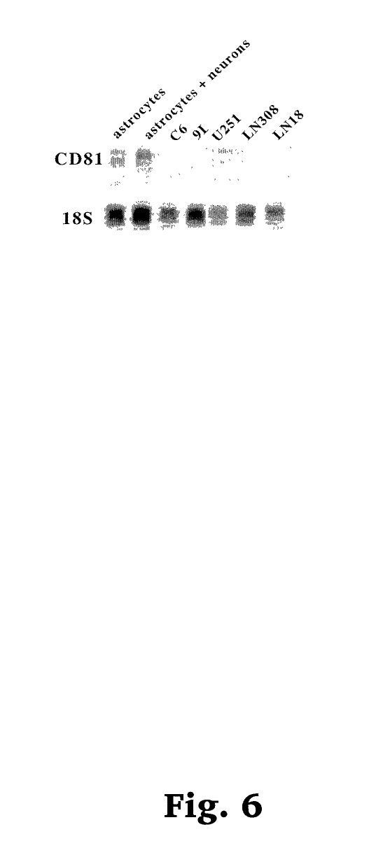

Tumorogenesis is a multistep phenomenon which contributes to a loss of growth control. To determine if CD81 might play a role in either astrocytic tumor progression or metastasis, the inventor assayed a number of astrocytoma cell lines for CD81 mRNA expression. Consistent with the immunofluorescence data, astrocytes expressed CD81 mRNA when isolated and cultured as a purified cell population. Upon co-culture with neuronal membranes, astrocyte expression of CD81 was upregulated, suggesting a positive feedback mechanism in maintaining astrocytes out of the cell cycle. In contrast, none of the astrocytoma cell lines assayed had detectable levels of CD81 message after 3 days of exposure (FIG. 6). There are likely to be a number of layers of neuron-regulated growth control, as no gross astrocytosis was observed in the CD81−/− animals when they were in the C57BL/6 background. However, when CD81−/− mice are bred into a BALB/c background, there is massive astrocytosis. Thus, there may be additional genetic components that interact with CD81 to regulate astrocyte proliferation in vivo. Nevertheless, the present data suggest that CD81 may play a role in astrocyte tumor progression.

4. Discussion

The establishment of the proper ratio of cell types within the mature CNS is not fully understood. While most neuronal populations are unable to re-enter the cell cycle after cellular differentiation, the same does not hold true for astrocytes. These cells are able to proliferate at any point in the life of the mammal, and do so under a variety of pathological conditions. However, in homeostasis, the number of astrocytes is remarkably conserved and maintained at a steady state (23, 24, 25). Previous work has shown that neuronal cells are a potent effector of astrocytic proliferative arrest and terminal differentiation. Moreover, these same mechanisms are likely to keep the astrocyte out of the cell cycle throughout life. While numerous candidate molecules have been proposed to be mediators of this activity, including NCAM (9), atrial natriuritic protein (21), astrotactin (4, 22), and endothelin 1 (26), none has withstood rigorous analysis.

Much of the analysis of neuron-astrocyte interactions has been modeled in vitro. When challenged with neurons under culture conditions, astrocytes withdraw from the cell cycle, and extend complex, GFAP-rich processes (11, 12, 29, 30). Herein, the inventor reports his recent findings that CD81 is a critical modulator of neuronal-mediated astrocyte differentiation and proliferative arrest. This conclusion is based on three separate, independent lines of experimental evidence. Antibody blocking, antigen competition, and genetic approaches all converge to suggest that CD81 is a critical part of this biology. Moreover, astrocytic tumor cell lines which were tested are CD81 deficient, further suggesting that CD81 is likely to play an important role in normal neuron-astrocyte biology. Importantly, the in vitro findings phenocopy in vivo events in some genetic backgrounds but not others, implying that CD81 either acts as a modifier of, or is modified by, additional genes.

Function-blocking antibodies are valuable tools for testing critical molecular interactions. The inventor has shown herein that Eat1, which binds to a discrete epitope located in the LEL of CD81, is able to ablate astrocytic responsiveness to co-culture with neurons, i.e., the astrocytes remain in the cell cycle, and fail to fully differentiate. In these studies, neurons still were able to adhere to the astrocytic cell surface, where they settled and extended prototypical, complex processes. The survival and differentiation of the neuronal cells suggest that there are multiple layers for neuron-astrocyte interactions, and that the astrocytes in these cultures were able to maintain the health of the neurons, even without full differentiation of the astrocytes. The requirement for astrocytes, or astrocyte-derived support, for granule cell survival and differentiation is well known (13). Therefore, these data suggest that CD81 activity in neuron-astrocyte interactions is specific to neuron-induced astrocyte differentiation.

Conformational changes, induced by binding of the various anti-CD81 mAbs to their cognate antigens, result in distinct astrocytic responses to neurons. While Eat2, which binds avidly to CD81, has no effect on function, the Eat1 antibody blocks interactions between CD81 and a heretofore unidentified partner. The idea of molecular cross-talk between CD81 and an unknown partner is supported by the observation that 2F7 increases the sensitivity of astrocytes to neuronal anti-proliferative signaling, suggesting that conformational changes in CD81 may have profound effects on its activity. This idea is further supported by evidence that shows that the 2F7 mAb is able to block thymocyte maturation (1). There has been at least one other tetraspanin, the Drosophila late bloomer gene, known to have a role in recognition between neural elements in development. Flies mutant in the late bloomer locus fail to make proper neuromuscular synapses in a timely fashion, suggesting a role in recognition of cellular elements in the fly nervous system (14). The tetraspanins, of which CD81 is a member, are thought to be molecular facilitators, bringing together partners within the plane of the membrane (19). A definitive answer to the molecular mechanism of CD81-mediated signaling between the astrocyte and the neuron awaits the identification of an astrocyte-expressed CD81 binding partner.

In general, studies using function-blocking antibodies are inherently limiting, because there are issues of non-specific steric inhibition for which it is difficult to control. The inventor has addressed this potential problem by competing for CD81 binding using soluble, GST-CD81 (LEL) fusion proteins. In this assay, the GST-CD81 (LEL) fusion proteins bind to the cell surface of neurons, but not astrocytes. The binding of neurons over astrocytes, and the failure of an irrelevant fusion protein to either bind or block function, suggests a specificity of binding, and raises the likelihood of a neuron-expressed CD81 receptor. More importantly, by competing with astrocyte-expressed CD81 for the putative neuronal CD81 receptors, these soluble GST-CD81 (LEL) proteins block neuron-induced astrocytic responses. These observations provide direct evidence that CD81 plays an important role in establishing neuron-induced astrocyte activity.

The final confirmation that CD81 plays a vital role in neuron-astrocyte biology was provided by establishing cultures from CD81 heterozygous and homozygous null mice. Using genetics to reduce or ablate CD81 expression, the inventor demonstrated a strict requirement for CD81 in neuron-induced astrocytic responses. The CD81 mice used in these studies were thoroughly backcrossed onto a C57BL/6 background. The +/− mice did not develop spontaneous astrocytic tumors, nor did they show signs of astrocytic hyperplasia, astrocytosis, or any detectable neurological abnormality. However, when the CD81 deletion was backcrossed onto the BALB/c background, a profound astrocytic hyperplasia resulted (E. Geisert et al., personal communication). This is a critical observation, as it demonstrates that there are modifiers of CD81 activity, which depend upon a genetic background to have an observable phenotype. It is notable that the association of a given gene product needs to be considered in the light of the surrounding genome, not en vacuo.

The implications of the current study extend beyond questions of regulating astrocyte cell number in homeostasis and injury. The inventor has examined a number of astrocytic tumor cell lines, all of which have severely attenuated levels of CD81 expression. While there is no evidence to suggest that CD81 is a classical tumor-suppressor gene, the absence of CD81 in these astrocytoma cell lines, taken together with CD81 function in normal neuron-astrocyte biology, raises the possibility that CD81 may be part of a tumor-suppressor cascade. The data presented here raise the possibility that mechanisms aimed at re-expressing CD81 in astrocytic tumors in situ may be of significant benefit for patients suffering from astrocytomas. Such an approach would be intended to limit the proliferative rate of the tumor cells in situ, thereby changing the otherwise lethal disease to a chronic ailment, and eliminating the neurologic damage of more conventional therapies. The invasive nature of glial tumors, as well as the neurological sequelae of extensive resection, make this type of approach appealing. Further studies, intended to elucidate the transcriptional regulation of CD81 in astrocytes, will provide insights into potential pharmacotherapeutics.

The data presented here clearly show the importance of CD81 in normal, neuron-induced astrocyte proliferative regulation. This observation reveals, to some extent, the mechanism(s) underlying the way in which the ratio of neurons and astrocytes is established and maintained in the adult CNS. Further delineation of the molecular mechanisms that control the dynamic interactions between these cell types is critical to the development of a more complete understanding of the means by which the mature nervous system achieves and maintains numerical homeostasis, and the way in which this balance may be restored when the nervous system moves out of equilibrium.

### REFERENCES

1. Boismenu et al., A role for CD81 in early T cell development. Science, 271:198-200, 1996.

2. Chomczynski and Sacchi, Single-step method of RNA isolation by acid guanidinium thiocyanate-phenol-chloroform extraction. Analytical Biochemistry, 162:156-59, 1987.

3. Easter et al., Initial tract formation in the mouse brain. Journal of Neuroscience, 13:285-99, 1993.

4. Edmondson et al., Astrotactin: a novel neuronal cell surface antigen that mediates neuron-astroglial interactions in cerebellar microcultures. Journal of Cell Biology, 106:505-17, 1988.

5. Flint et al., Characterization of hepatitis C virus E2 glycoprotein interaction with a putative cellular receptor, CD81. Journal of Virology, 73:6235-44, 1999.

6. Frisen et al., Rapid, widespread, and longlasting induction of nestin contributes to the generation of glial scar tissue after CNS injury. Journal of Cell Biology, 31:453-64, 1995.

7. Fujita, S., Quantitative analysis of cell proliferation and differentiation in the cortex of the postnatal mouse cerebellum. Journal of Cell Biology, 32:277-87, 1967.

8. Geisert et al., Astrocyte growth, reactivity, and the target of the antiproliferative antibody, TAPA. J. Neurosci., 16:5478-87, 1996.

9. Gimenez y Ribotta et al., Oxysterol (7 beta-hydroxycholesteryl-3-oleate) promotes serotonergic reinnervation in the lesioned rat spinal cord by reducing glial reaction. Journal of Neuroscience Research, 41:79-95, 1995.

10. Hatten, M. E., Riding the glial monorail: a common mechanism for glial-guided neuronal migration in different regions of the developing mammalian brain. Trends in Neuroscience, 13:179-84, 1990.

11. Hatten and Liem, Astroglial cells provide a template for the positioning of developing cerebellar neurons in vitro. Journal of Cell Biology, 90:622-30, 1981.

12. Hatten et al., Neuron-astroglial interactions in vitro and their implications for repair of CNS injury. Central Nervous System Trauma, 1:15-27, 1984.

13. Hatten and Shelanski, Mouse cerebellar granule neurons arrest the proliferation of human and rodent astrocytoma cells in vitro. Journal of Neuroscience, 8:1447-53, 1988.

14. Kopczynski et al., A neural tetraspanin, encoded by late bloomer, that facilitates synapse formation, Science, 271:1867-70, 1996.

15. Latov et al., Fibrillary astrocytes proliferate in response to brain injury: a study combining immunoperoxidase technique for glial fibrillary acidic protein and radioautography of tritiated thymidine. Developmental Biology, 72:381-84, 1979.

16. Maecker et al., CD81 on B cells promotes interleukin 4 secretion and antibody production during T helper type 2 immune responses. Proc. Natl Acad. Sci. USA, 95:2458-62, 1998.

17. Maecker and Levy, Normal lymphocyte development but delayed humoral immune response in CD81-null mice. J. Exp. Med., 185:1505-10, 1997.

18. Maecker et al., Differential expression of murine CD81 highlighted by new anti-mouse CD81 monoclonal antibodies. Hybridoma, 19:15-22, 2000.

19. Maecker et al., The tetraspanin superfamily: molecular facilitators. FASEB J., 11:428-42, 1997.

20. Miyazaki et al., Normal development but differentially altered proliferative responses of lymphocytes in mice lacking CD81. EMBO J., 16:4217-25, 1997.

21. Pedram et al., Astrocyte progression from G1 to S phase of the cell cycle depends upon multiple protein interaction. J.

Biol. Chem., 273:13966-72, 1998.

22. Stitt and Hatten, Antibodies that recognize astrotactin block granule neuron binding to astroglia. Neuron, 5:639-49, 1990.

23. Sturrock, R. R., Histogenesis of the anterior limb of the anterior commissure of the mouse brain. 3. An electron microscopic study of gliogenesis. Journal of Anatomy, 117:37-53, 1974.

24. Sturrock, R. R., Histogenesis of the anterior limb of the anterior commissure of the mouse brain. III. A quantitative study of changes in the glial population with age. Journal of Anatomy, 117:17-25, 1974.

25. Sturrock, R. R., Histogenesis of the anterior limb of the anterior commissure of the mouse brain. II. A quantitative study of pre- and postnatal mitosis. Journal of Anatomy, 117:27-35, 1974.

25. Teixeira et al., Differential regulation of cyclin D1 and D3 expression in the control of astrocyte proliferation induced by endothelin-1. Journal of Neurochemistry, 74:1034-40, 2000.

26. Tsitsikov et al., Impaired CD19 expression and signaling, enhanced antibody response to type II T independent antigen and reduction of B-1 cells in CD81-deficient mice. Proc. Natl Acad. Sci. USA, 94:10844-49, 1997.

27. Weinstein, D. E., The Culture of Primary Astrocytes. In Current Protocols in Neuroscience, E. Crawley, C. Gerfen, R. McKay, M. Rogawski, D. Sibley, and P. Skolnick, eds. (New York: Wiley and Son), 1997.

28. Weinstein et al., C17, a retrovirally immortalized neuronal cell line, inhibits the proliferation of astrocytes and astrocytoma cells by a contact-mediated mechanism. Glia, 3:130-39, 1990.

29. Weinstein et al., Suppression by antisense mRNA demonstrates a requirement for the glial fibrillary acidic protein in the formation of stable astrocytic processes in response to neurons. Journal of Cell Biology, 112:205-13, 1991.

30. Beers and Berkow, eds., The Merck Manual of Diagnosis and Therapy, 17^{th} ed. (Whitehouse Station, NJ: Merck Research Laboratories, 1999) 1395-1398, 1442-48.

All publications mentioned hereinabove are hereby incorporated in their entireties. While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A method for inhibiting proliferation of astrocytes, comprising contacting astrocytes with an amount of CD81 effective to inhibit proliferation of astrocytes.

2. The method of claim 1, wherein astrocytes are contacted with CD81 by introducing CD81 protein into membranes of the astrocytes.

3. The method of claim 1, wherein astrocytes are contacted with CD81 by introducing into the astrocytes a nucleic acid encoding CD81, in a manner permitting expression of CD81.

4. The method of claim 3, wherein the nucleic acid is introduced by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, and naked DNA transfer.

5. The method of claim 1, wherein the contacting is effected in vivo.

6. The method of claim 5, wherein the contacting is effected in vivo in a mammal.

7. The method of claim 6, wherein the mammal is a human.

8. The method of claim 7, wherein the human has a condition associated with a defect in astrocyte proliferation.

9. The method of claim 8, wherein the defect in astrocyte proliferation is astrocytosis.

10. The method of claim 7, wherein astrocytes are contacted with CD81 by introducing CD81 protein into membranes of the astrocytes.

11. The method of claim 7, wherein astrocytes are contacted with CD81 by introducing into the astrocytes a nucleic acid encoding CD81, in a manner permitting expression of CD81.

12. The method of claim 11, wherein the nucleic acid is introduced by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, and naked DNA transfer.

13. A method for inhibiting proliferation of astrocytic tumor cells, comprising contacting astrocytic tumor cells with an amount of CD81 effective to inhibit proliferation of astrocytic tumor cells.

14. The method of claim 13, wherein astrocytic tumor cells are contacted with CD81 by introducing CD81 protein into membranes of the astrocytic tumor cells.

15. The method of claim 13, wherein astrocytic tumor cells are contacted with CD81 by introducing into the astrocytic tumor cells a nucleic acid encoding CD81, in a manner permitting expression of CD81.

16. The method of claim 15, wherein the nucleic acid is introduced by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, and naked DNA transfer.

17. The method of claim 3, wherein the contacting is effected in vivo.

18. The method of claim 17, wherein the contacting is effected in vivo in a mammal.

19. The method of claim 18, wherein the mammal is a human.

20. The method of claim 19, wherein the human has a condition associated with proliferation of astrocytic tumor cells.

21. The method of claim 20, wherein the condition associated with proliferation of astrocytic tumor cells is an astrocytoma.

22. The method of claim 19, wherein astrocytic tumor cells are contacted with CD81 by introducing CD81 protein into membranes of the astrocytic tumor cells.

23. The method of claim 19, wherein astrocytic tumor cells are contacted with CD81 by introducing into the astrocytic tumor cells a nucleic acid encoding CD81, in a manner permitting expression of CD81.

24. The method of claim 23, wherein the nucleic acid is introduced by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, and naked DNA transfer.

25. A method for treating a condition associated with a defect in astrocyte proliferation in a subject in need of treatment, comprising administering to the subject an amount of CD81 effective to treat the condition associated with a defect in astrocyte proliferation.

26. The method of claim 25, wherein the condition associated with a defect in astrocyte proliferation is astrocytosis.

27. The method of claim 25, wherein CD81 is administered orally, parenterally, or transdermally.

28. A method for treating a condition associated with proliferation of astrocytic tumor cells in a subject in need of treatment, comprising administering to the subject an amount of CD81 effective to treat the condition associated with proliferation of astrocytic tumor cells.

29. The method of claim 28, wherein the condition associated with proliferation of astrocytic tumor cells is an astrocytoma.

30. The method of claim 28, wherein CD81 is administered orally, parenterally, or transdermally.

31. A method for determining whether a subject has an astrocytoma, comprising assaying for CD81 expression a diagnostic sample of cells of astrocytic lineage of the subject, wherein no detection of expression of CD81 in cells of astrocytic lineage of the subject is diagnostic of an astrocytoma.

32. The method of claim 31, wherein the diagnostic sample of cells of astrocytic lineage of the subject is assayed in vitro or in vivo.

33. A method for assessing the efficacy of astrocytoma therapy in a subject who has undergone or is undergoing treatment for an astrocytoma, comprising assaying for CD81 expression a diagnostic sample of cells of astrocytic tumor cells of the subject, wherein no detection of expression of CD81 in astrocytic tumor cells of the subject is indicative of unsuccessful astrocytoma therapy.

34. The method of claim 33, wherein the diagnostic sample of cells of astrocytic lineage of the subject is assayed in vitro or in vivo.
